(19) United States
(12) Patent Application Publication
(10) Pub. No.: US 2007/0009899 A1
(43) Pub. Date: Jan. 11, 2007

(54) Nucleic acid arrays for detecting gene expression in animal models of inflammatory diseases

(76) Inventor: William Martin Mounts, Andover, MA (US)

Correspondence Address: NIXON PEABODY, LLP, 401 9TH STREET, NW, SUITE 900, WASHINGTON, DC, 20004-2128, US

(21) Appl. No.: 10/956,160

(22) Filed: Oct. 4, 2004

Related U.S. Application Data
(60) Provisional application No. 60/507,481, filed on Oct. 2, 2003.

(51) Int. Cl. C12Q 1/68; C07H 21/04; C12M 1/34

(52) U.S. Cl. 435/6; 435/287.2; 536/24.3

(57) ABSTRACT

The present invention provides nucleic acid arrays and methods of using the same for detecting gene expression in animal models of osteoarthritis or other inflammatory diseases. The nucleic acid arrays of the present invention comprise polynucleotide probes for genes that are differentially expressed in osteoarthritis-affected cartilage tissues as compared to non-osteoarthritic cartilage tissues. In one embodiment, a nucleic acid array of the present invention comprises a plurality of polynucleotide probe sets, each of which is capable of hybridizing under stringent or nucleic acid array hybridization conditions to a different respective tiling sequence selected from Table C, or the complement thereof.

### RELATED APPLICATIONS

This application claims benefit and incorporates by reference the entire disclosure of U.S. Provisional Application Ser. No. 60/507,481 filed Oct. 2, 2003.

All materials on the compact discs labeled “Copy 1” and “Copy 2” are incorporated herein by reference in their entireties. Each of the compact discs includes the following files: Table B1.txt (318 KB, created Oct. 1, 2004), Table B2.txt (1,016 KB, created Oct. 1, 2004), Table C.txt (1,335 KB, created Sep. 14, 2004), Table D.txt (183 KB, created Sep. 14, 2004), Table E.txt (1,388 KB, created Sep. 14, 2004), Table F.txt (11,546 KB, created Sep. 14, 2004), Table I.txt (11,587 KB, created Sep. 14, 2004), and Sequence Listing.ST25.txt (54,107 KB, created Sep. 29, 2004).

LENGTHY TABLES FILED ON CD
The patent application contains a lengthy table section. A copy of the table is available in electronic form from the USPTO web site (http://seqdata.uspto.gov/?pageRequest=docDetail&DocID=US20070009899A1) An electronic copy of the table will also be available from the USPTO upon request and payment of the fee set forth in 37 CFR 1.19(b)(3).

### TECHNICAL FIELD

This invention relates to nucleic acid arrays and methods of using the same for detecting gene expression in animal models of osteoarthritis or other inflammatory diseases.

### BACKGROUND

Osteoarthritis is one of the most common diseases of the elderly. It mostly affects the weight-bearing joints such as spine, knees and hips, but thumb and finger joints may also be affected. Osteoarthritis is mainly a disease of “wear and tear.” Repetitive mechanical injury of the cartilage eventually results in loss of cartilage and damage to joint surfaces and adjacent bone. Inflammatory cells then invade the damaged joints, causing pain, swelling and stiffness of the joints. The repetitive mechanical injury also leads to pathological changes that are characterized by the loss of proteoglycans and collagen from the cartilage matrix.

Animal models of osteoarthritis, such as canine models, are currently used for studying the pathogenesis of cartilage degeneration. In addition, these models are used for evaluating new drugs or therapies for treating osteoarthritis. Typically, osteoarthritis in an animal model can be either spontaneous, or surgically induced using procedures such as medial partial or total meniscectomy or anterior cruciate ligament transection. Animal models of osteoarthritis mimic human osteoarthritis. They provide a broad spectrum of end-points for evaluating joint damage. Moreover, animal models are site-specific and reproducible. The onset and progress of osteoarthritis can be readily monitored in the animal models.

### SUMMARY OF THE INVENTION

The present invention provides nucleic acid arrays and methods of using the same for detecting gene expression in animal models of osteoarthritis or other inflammatory diseases. Preferred animal models include canine animals, such as dogs. Other animal models, such as mice, rats, rabbits, hamsters, and guinea pigs, can also be used.

In one aspect, the nucleic acid arrays of the present invention comprise at least one polynucleotide probe capable of hybridizing under stringent and/or nucleic acid array hybridization conditions to a gene which is differentially expressed in osteoarthritic cartilage cells relative to non-osteoarthritic cartilage cells. In many cases, the osteoarthritic and non-osteoarthritic cartilage cells are derived from the same species, such as dog (Canis familiaris) or another canine species. The osteoarthritic and non-osteoarthritic cartilage cells can be prepared from the same animal. For instance, the osteoarthritic cartilage cells can be prepared from one leg of an animal that is induced with osteoarthritis, while the other matching limb in the animal is kept unaffected to produce donor-matched non-osteoarthritic cartilage cells. The osteoarthritic and non-osteoarthritic cartilage cells can also be prepared from different animals, such as from an osteoarthritic animal and a non-osteoarthritic animal, respectively. As used herein, a polynucleotide probe can hybridize to a gene if the probe can hybridize to an mRNA, a cDNA or a codon sequence of the gene, or the complement thereof.

In one embodiment, a nucleic acid array of the present invention includes at least 1, 2, 3, 4, 5, 10, 50, 100, 500, 1,000, or more polynucleotide probe sets. Each of these probe sets is capable of hybridizing under stringent and/or nucleic acid array hybridization conditions to a different respective gene which is differentially expressed in osteoarthritic cartilage cells relative to non-osteoarthritic cartilage cells. As used herein, a probe set can hybridize to a gene if each probe in the probe set can hybridize to the gene. By “different respective,” it means that each probe set in a group of probe sets can hybridize to a gene that is different from those to which other probe sets in the group hybridize. Each probe set can include any number of probes, such as 2, 5, 10, 15, 20, 25, or more. In one example, each probe set employed in the present invention includes at least 12 polynucleotide probes.

In another embodiment, a nucleic acid array of the present invention includes at least 1, 2, 3, 4, 5, 10, 50, 100, 500, 1,000, or more polynucleotide probe sets, each of which is capable of hybridizing under stringent and/or nucleic acid array hybridization conditions to a different respective gene whose average expression level in osteoarthritic cartilage cells is higher or substantially higher than that in non-osteoarthritic cartilage cells. In still another embodiment, the nucleic acid array further comprises at least 1, 2, 3, 4, 5, 10, 50, 100, 500, 1,000, or more polynucleotide probe sets, each of which is capable of hybridizing under stringent and/or nucleic acid array hybridization conditions to a different respective gene whose average expression level in non-osteoarthritic cartilage cells is higher or substantially higher than that in osteoarthritic cartilage cells.

In a further embodiment, a nucleic acid array of the present invention includes at least 1, 2, 3, 4, 5, 10, 50, 100, 500, 1,000, or more polynucleotide probes or probe sets, each of which is capable of hybridizing under stringent and/or nucleic acid array hybridization conditions to a different respective tiling sequence selected from Table C, or the complement thereof. In one example, the polynucleotide probes or probe sets can hybridize under stringent and/or nucleic acid array hybridization conditions to respective tiling sequences (or the complements thereof) that have pattern values of 010, 011, 100 or 101. In another example, the nucleic acid array includes at least one polynucleotide probe for each tiling sequence selected from Table C, or the complement thereof. In still another example, the nucleic acid array includes each and every polynucleotide probe selected from Table F. In still yet another example, the nucleic acid array includes a perfect mismatch probe for each perfect match probe stably attached to the nucleic acid array.

In another aspect, the present invention provides methods of screening for drug candidates capable of modulating expression of genes that are differentially expressed in osteoarthritic cartilage cells relative to non-osteoarthritic cartilage cells. In one example, the methods comprise the steps of:

(a) preparing a first nucleic acid sample from a vertebrate affected by osteoarthritis;

(b) hybridizing the first nucleic acid sample to a first nucleic acid array of the present invention;

(c) detecting a first set of hybridization signals;

(d) treating the vertebrate with a candidate drug;

(e) repeating steps (a)-(c) with a second nucleic acid sample from the treated vertebrate and a second nucleic acid array identical to the first array to obtain a second set of hybridization signals; and

(f) comparing the first and second sets of hybridization signals, where any change in expression level of at least one gene differentially expressed in osteoarthritic cartilage cells relative to non-osteoarthritic cartilage cells identifies the candidate drug as one that modulates expression of that gene. In many cases, the vertebrate is a canine animal (e.g., a dog) or a human, and the first and second nucleic acid samples are prepared from cartilage tissues of the canine animal or human. Other vertebrates or tissues (e.g., body fluids) can also be analyzed according to the present invention.

In another embodiment, the methods of the present invention comprise the steps of:

(a) preparing a first nucleic acid sample from a cartilage cell or tissue affected by osteoarthritis;

(b) hybridizing the first nucleic acid sample to a first nucleic acid array of the present invention;

(c) detecting a first set of hybridization signals;

(d) treating the cell or tissue with a candidate drug;

(e) repeating steps (a)-(c) with a second nucleic acid sample from the treated cell or tissue and a second nucleic acid array identical to the first array to obtain a second set of hybridization signals; and

(f) comparing the first and second sets of hybridization signals, where any change in expression level of at least one gene differentially expressed in osteoarthritic cartilage cells relative to non-osteoarthritic cartilage cells identifies the candidate drug as one that modulates expression of that gene.

The present invention also features methods for detecting gene expression in a sample of interest. The methods comprise the steps of hybridizing nucleic acid molecules prepared from the sample of interest to a nucleic acid array of the present invention; and detecting hybridization signals on the nucleic acid array.

In addition, the present invention features methods for making nucleic acid arrays. The methods comprise the steps of selecting a plurality of polynucleotides, each of which is capable of hybridizing under stringent or nucleic acid array hybridization conditions to a different respective gene which is differentially expressed in osteoarthritic cartilage cells relative to non-osteoarthritic cartilage cells; and attaching the plurality of polynucleotide probes to one or more substrate supports. In many cases, the osteoarthritic and non-osteoarthritic cartilage cells are derived from the same species.

Furthermore, the present invention features probe arrays for the detection of protein levels in animal models of osteoarthritis or other inflammatory diseases. Each of these probe arrays comprises probes or probe sets capable of specifically binding to protein products of genes that are differentially expressed in osteoarthritic cartilage cells relative to non-osteoarthritic cartilage cells. In one embodiment, a probe array of the present invention comprises a plurality of antibodies, each of which can specifically bind to a protein product of a gene that encodes a tiling sequence selected from Table C and having a pattern value of 010, 011, 100 or 101.

The present invention also features polynucleotide collections. In many embodiments, the polynucleotide collections of the present invention comprise at least one polynucleotide capable of hybridizing under stringent or nucleic acid array hybridization conditions to a parent sequence selected from SEQ ID NOs: 1-12,167, or the complement thereof. In one example, a polynucleotide collection of the present invention includes at least one tiling sequence selected from Table C, or the complement thereof. In another example, a polynucleotide collection of the present invention comprises (1) at least one polynucleotide capable of hybridizing under stringent and/or nucleic acid array hybridization conditions to a tiling sequence selected from Table C and having a pattern value of 010, or the complement thereof; and (2) at least another polynucleotide capable of hybridizing under stringent and/or nucleic acid array hybridization conditions to a tiling sequence selected from Table C and having a pattern value of 100, or the complement thereof. In still another example, a polynucleotide collection of the present invention includes at least one polynucleotide comprising a sequence selected from SEQ ID NOs: 1-12,167.

Other features, objects, and advantages of the present invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### BRIEF DESCRIPTION OF THE DRAWING

The drawing is provided for illustration, not limitation.

FIG. 1 represents an Eisen cluster of transcriptional profiling data generated with a nucleic acid array of the present invention.

### DETAILED DESCRIPTION

I. Definitions

“Nucleic acid array hybridization conditions” refer to the temperature and ionic conditions that are normally used in nucleic acid array hybridization. These conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer preferably is between 6.5 and 6.7. The wash buffer is 6× SSPET. 6× SSPET contains 0.9 M NaCl, 60 mM NaH₂PO₄, 6 mM EDTA, and 0.005% Triton X-100. Under more stringent nucleic acid array hybridization conditions, the wash buffer can contain 100 mM MES, 0.1 M [Na⁺], and 0.01% Tween 20.

The frequency of occurrence of an mRNA transcript is “substantially higher” in one tissue than in another tissue if the molar concentration of the mRNA transcript relative to the total mRNA in the former tissue is at least 1.5-fold of that in the latter tissue. For instance, the molar concentration of the mRNA transcript/molecule relative to the total mRNA in the former tissue can be at least 2-fold, 3-fold, 4-fold, 5-fold, 10-fold, 20-fold or more of that in the latter tissue. In one instance, the mRNA transcript is detectable in the former tissue but not in the latter tissue. In another instance, the mRNA transcript is more readily identifiable using 3′ sequence reads from a cDNA library prepared from the former tissue than that prepared from the latter tissue.

“Stringent conditions” are at least as stringent as, for example, conditions G-L shown in Table A. In certain embodiments of the present invention, highly stringent conditions A-F can be used. In Table A, hybridization is carried out under the hybridization conditions (Hybridization Temperature and Buffer) for about four hours, followed by two 20-minute washes under the corresponding wash conditions (Wash Temp. and Buffer).

TABLE A
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown | | | | |
| # sequence, the hybrid length is assumed to be that of the hybridizing polynucleotide. When polynucleotides of known sequence are hybridized, the hybrid length can be determined by | | | | |
| # aligning the sequences of the polynucleotides and identifying the region or regions of optimal sequence complementarity. | | | | |
| ^{H}SSPE (1x SSPE is 0.15 M NaCl, 10 mM NaH₂PO₄, and 1.25 mM EDTA, pH 7.4) can be substituted for SSC (1x SSC is 0.15 M NaCl and 15 mM sodium citrate) in the | | | | |
| # hybridization and wash buffers. | | | | |
| T_{B}*-T_{R}*: The hybridization temperature for hybrids anticipated to be less than 50 base pairs in length should be 5-10° C. less than the melting temperature (Tₘ) of the hybrid, | | | | |
| # where Tₘ is determined according to the following equations. For hybrids less than 18 base pairs in length, Tₘ(° C.) = 2(# of A + T bases) + 4(# of G + C bases). For hybrids between 18 | | | | |
| # and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions in | | | | |
| # the hybridization buffer (Na⁺ for 1xSSC = 0.165 M). | | | | |

Various aspects of the invention are described in further detail in the following sections or subsections. The use of sections and subsections is not meant to limit the invention; each section and subsection may apply to any aspect of the invention.

II. The Invention

The nucleic acid arrays of the present invention comprise polynucleotide probes for expression profiling of genes that are differentially expressed in osteoarthritis-affected cartilage tissues as compared to non-osteoarthritic cartilage tissues. The polynucleotide probes can be derived from cartilage tissues of any animal model of osteoarthritis. Suitable animal models of osteoarthritis include, but are not limited to, canines, rodents, rabbits, and primates. Preferred animal models include dogs, mice, rats, rabbits, hamsters, and guinea pigs. Osteoarthritis can naturally occur in some of these animal models, such as mice, hamsters, or guinea pigs. Osteoarthritis can also be surgically induced in animals like dogs and rabbits.

The nucleic acid arrays of the present invention can further include probes for expression profiling of other genes that are not associated with osteoarthritis. These probes can be derived from a variety of sources, including publicly accessible databases, such as GenBank. These probes can also be derived from cDNA libraries prepared from numerous animal tissues.

The nucleic acid arrays of the present invention can be used to identify or validate novel therapeutic targets for osteoarthritis. The nucleic acid arrays of the present invention can also be used to screen for potential drug candidates or evaluate new therapies for treating osteoarthritis. In addition, the nucleic acid arrays of the present invention can be used to monitor the global gene expression in animal models of osteoarthritis or other inflammatory diseases.

The following sections/subsections focus on the preparation of nucleic acid arrays suitable for the detection of gene expression in Canis familiaris. As appreciated by those skilled in the art, the same methodology can be readily adapted to the making of nucleic acid arrays suitable for the detection of gene expression in other animal disease models.

A. Collection of mRNA/cDNA Sequences of Canis familiaris

mRNA/cDNA sequences of Canis familiaris can be collected or derived from a variety of sources, such as GenBank and TIGR (The Institute for Genome Research). These publicly accessible sequence databases frequently include a large number of ESTs, cDNAs, and other transcribed or transcribable sequences of a species of interest. In addition, these sequence databases contain a large amount of genomic sequences. Open reading frames (ORFs) in these genomic sequences can be predicted or isolated using methods known in the art. Suitable methods for this purpose include, but are not limited to, GeneMark (provided by the European Bioinformatics Institute), Glimmer (provided by TIGR), and ORF Finder (provided by the National Center for Biotechnology Information).

mRNA/cDNA sequences can also be obtained by sequencing cDNA clones isolated from cDNA libraries. Suitable cDNA libraries can be prepared from any tissue of Canis familiaris. In one embodiment, a cDNA library is prepared from a cartilage tissue. The cartilage tissue can be either non-osteoarthritic, or affected by osteoarthritis. Exemplary cartilage tissues suitable for the present invention include hyaline cartilage, elastic cartilage, fibrous cartilage, and articular cartilage. In one specific example, the cartilage tissue is isolated from the large joints of Canis familiaris.

Methods for constructing a cDNA library are well known in the art. In standard methods, the mRNA in the cells of interest, such as cartilage cells, is isolated by virtue of the presence of a polyadenylated (polyA) tail present at the 3′ end of the mRNA. The polyA tail binds to a resin conjugated with oligo-dT (oligo-dT chromatography). The purified mRNA is then copied into cDNA using a reverse transcriptase and a primer under conditions sufficient for the first strand cDNA synthesis to occur. Although both random and specific primers can be employed, in many embodiments the primer is an oligo dT primer that provides for hybridization to the polyA tail in the mRNA. The oligo dT primer is sufficiently long to provide for efficient hybridization to the polyA tail. Typically, the oligo dT primer ranges from 10 to 25 nucleotides in length, such as from 12 to 18 nucleotides in length. Additional reagents, such as dNTPs, buffering agents (e.g. TrisCl), cationic sources (monovalent or divalent, e.g. KCl, MgCl₂), and sulfhydril reagents (e.g. dithiothreitol), can also be included in the reaction.

A variety of enzymes, usually DNA polymerases possessing reverse transcriptase activity, can be used for the first strand cDNA synthesis. Examples of suitable DNA polymerases include the DNA polymerases derived from thermophilic bacteria, archaebacteria, retroviruses, yeasts, Neurosporas, Drosophilas, primates, or rodents. In one embodiment, the DNA polymerase is derived from Moloney murine leukemia virus (M-MLV), human T-cell leukemia virus type I (HTLV-I), bovine leukemia virus (BLV), Rous sarcoma virus (RSV), human immunodeficiency virus (HIV), Thermus aquaticus (Taq), Thermus thermophilus (Tth), or avian reverse transcriptase. M-MLV reverse transcriptase that lacks RNaseH activity can also be used. See, for example, U.S. Pat. No. 5,405,776, which is incorporated herein by reference.

The order in which the reagents are combined can be modified as desired. In one protocol, all reagents except for the reverse transcriptase are combined on ice, and then the reverse transcriptase is added at around 40° C. Following the addition of the reverse transcriptase, the temperature of the reaction mixture can be raised to 37° C., followed by incubation for a period of time sufficient for the primer extension to form the first strand of cDNA. The primer extension starts at the 3′ end of the mRNA and proceeds towards the 5′ end. The incubation period can take about 1 hour.

Second strand cDNA synthesis is then performed. Linkers are added to the ends of the double stranded cDNA to allow for its package into virus or cloning into plasmids/vectors. At this stage, the cDNA is in a form that can be propagated. The linkers or the primers can include rare restriction enzyme sites, such as Not I and/or Pac I, to facilitate the cloning of the cDNA into plasmids/vectors. Suitable plasmids/vectors for subcloning cDNA molecules include, for example, the pT7T3-Pac vector (a modified pT7T3 vector, Pharmacia), the pSPORT 1 vector (Invitrogen), and the various lambda cDNA library vectors provided by Stratagene (La Jolla, Calif.).

In one embodiment, mRNA is purified through its unique 5′-cap structure. The 5′-cap structure of eukaryotic mRNA includes m7GpppN, where N can be any nucleotide. Resins conjugated with a 5′-cap binding agent can be used to purify mRNA. Suitable 5′-cap binding agents include, but are not limited to, the eIF-4E/eIF-4G fusion protein disclosed in U.S. Pat. No. 6,326,175, which is incorporated herein by reference. The first strand cDNA synthesis can be performed using any conventional protocol. Following the first strand cDNA synthesis, the resultant mRNA/DNA duplex is contacted with an RNase to degrade single stranded RNA but not RNA complexed to DNA. Suitable RNases for this purpose include RNase Ti from Aspergillus orzyae, RNase I, and RNase A. The conditions and duration of incubation during this step can vary depending on the specific nuclease employed. Generally, the incubation temperature is between about 20° C. to 37° C., and the incubation time lasts from about 10 to 60 min.

Nuclease treatment produces blunt-ended mRNA/DNA duplexes. The mRNA/DNA hybrids that include the unique 5′-cap structure can be isolated using resins conjugated with the eIF-4E/eIF-4G fusion protein. Following isolation, the nucleic acids can be further processed, including release from the resins and production of double stranded cDNA. The double stranded cDNA is then subcloned into appropriate plasmids/vectors to create a cDNA library.

In a preferred embodiment, the cDNA library is prepared using the CloneMinerm™ cDNA Library Construction Kit provided by Invitrogen (Carlsbad, Calif.). The CloneMiner Kit uses a modified reverse transcriptase and a biotin-attB-oligo(dT) primer to synthesize the first strand of cDNA. The modified reverse transcriptase has reduced RNAase H activity, thereby decreasing RNA degradation during the first strand synthesis. The second strand of cDNA is then synthesized using E. coli DNA polymerase I, and an attB adaptor is added to the 5′ end of the double stranded cDNA.

The att sites, including the attB and attP sites, are components of the lambda recombination system. Recombination between the attB and attP sites swaps the sequences located therebetween. The CloneMiner destination vectors contain the attP sites which flank the ccdB gene. The ccdB gene inhibits the growth of most E. coli strains. Recombination between the attB-flanked cDNA product and the destination vectors replaces the ccdb gene with the cDNA product, thereby removing the inhibitory effect of the ccdB gene and allowing negative selection of the recombinant vector that contains the cDNA insert. The selected recombinant vector is then transformed into competent E. coli cells to produce a cDNA library. The cDNA library prepared using the CloneMiner cDNA Library Construction Kit preferably includes at least 5×10⁶, 1×10⁷, 5×10⁷ or more primary clones.

According to the CloneMiner user's manual, cDNA can be either radiolabeled or non-radiolabeled during its synthesis. Radiolabeling facilitates the measurement of cDNA yield and overall quality of the first strand cDNA synthesis. For instance, if [α-³²P]dCTP is used to monitor the first strand reaction, the percent incorporation of [α-³²P]dCTP preferably is no less than 10%. More preferably, the percent incorporation of [α-³²P]dCTP is about 20-50%.

In addition, cDNA can be size fractionated before being subcloned into the destination vectors. Suitable methods for size fractionation include, but are not limited to, column chromatography and gel electrophoresis. The final cDNA yield after size fractionation and subsequent ethanol precipitation preferably is no less than 30-40 ng. In some cases, at least 50, 75, 100, 150, 200 ng or more cDNA is used for subcloning.

The cDNA libraries used in the present invention can be prepared from any tissue of Canis familiaris. In one preferred embodiment, the cDNA library is prepared from osteoarthritis-affected cartilage tissues. Cartilage samples can be surgically removed from the knees of osteoarthritic dogs, and then homogenized and extracted for mRNA. Suitable agents for mRNA extraction include, but are not limited to, guanidine isothiocyanate/acidic phenol method, the TRIZOL™ Reagent (Invitrogen), or the Micro-FastTrack® 2.0 or FastTrack™ 2.0 mRNA Isolation Kits (Invitrogen). Alternatively, cartilage cells (e.g. chondrocytes) can be first dissociated from the cartilage samples, and then extracted for mRNA. The extracted mRNA is subsequently purified based on its unique 3′ or 5′ structure. The mRNA extraction and purification steps are preferably conducted under conditions where the RNase activities are minimized. The quality of the purified mRNA can be monitored using agarose/ethidium bromide gel electrophoresis. The amount of the purified mRNA can range from 0.5 to 10 μg. Preferably, at least 2 μg of purified mRNA is used for the construction of a cDNA library. In one embodiment, 1 to 5 μg of mRNA is used for preparing a cDNA library containing 10⁶ to 10⁷ primary clones in E. coli.

Similar methods can be used to prepare cDNA libraries from non-osteoarthritic cartilage tissues.

Canis familiaris cDNA sequences can be readily obtained from these cartilage cDNA libraries. In standard methods, individual cDNA clones in these libraries are isolated. Vectors containing the cDNA inserts are purified, followed by the sequencing of the cDNA inserts. The sequencing primers can be designed based on the common vector sequences adjacent to the 5′ or 3′ end of the cDNA inserts.

In one embodiment, Canis familiaris cDNA sequences are collected using the 3′ sequence reads from an osteoarthritis-affected cartilage cDNA library as well as an osteoarthritis-free cartilage cDNA library. Both libraries are prepared by using oligo-d(T) primers for first strand cDNA synthesis. Both libraries are constructed such that the frequency of occurrence of each cDNA clone in each cDNA library is proportional to the molar concentration of the corresponding mRNA in the cartilage tissue from which that cDNA library is derived. The frequency of occurrence of each cDNA clone also correlates with the chance of that cDNA clone being identified in the cDNA library. Thus, the easiness of a cDNA clone being identified in the cDNA library can indicate that the corresponding mRNA transcript has a relatively high level of expression in the cartilage tissue from which the cDNA library is derived.

The 3′ sequence reads from the cartilage libraries can be further edited before being used for other purposes. For instance, the vector sequences at the 5′ end of the sequence read product can be removed or masked out. This process may be carried out automatically, such as by employing a screening algorithm, or conducted manually. In addition to trimming the 5′ end, the 3′ end of the sequence read product can also be trimmed. Typically, the quality of the sequence read may decrease as it moves towards the 3′ end of the sequence. Thus, by trimming the 3′ end, the overall quality and accuracy of the eventual sequence will be improved.

The edited 3′ sequence reads from both the osteoarthritic cartilage library and the osteoarthritis-free cartilage library, together with the Canis familiaris sequences obtained from GenBank, can be clustered to identify highly homologous cDNA sequences. Suitable clustering algorithms for this purpose include, but are not limited to, the CAT (cluster and alignment tool) software package provided by DoubleTwist. See Clustering and Alignment Tools User's Guide (DoubleTwist, Inc., 2000).

The CAT program can reduce the redundancy, as well as mask low-complexity regions of the input sequence set. The resulting sequence set derived from CAT contains two distinct groups of sequences. The first group is a set of consensus sequences derived from multiple sequence alignment produced for CAT clusters containing more than one sequence. These multi-sequence clusters may include single transcripts represented in the input sequence set numerous times. The second group is a set of exemplar sequences that do not cluster with any other CAT cluster. The consensus and exemplar sequences can be generated such that any base ambiguity would be identified with the respective IUPAC (International Union of Pure and Applied Chemistry) base representation, which is identical to the WIPO Standard ST.25 (1998).

In a small number of cases, the multi-sequence clusters contain a large number of sequences due to clustering artifacts (e.g., highly homologous genes or domains). In these cases, through more stringent clustering parameters, the large clusters are re-clustered. In addition, the consensus sequences can be manually curated to verify cluster membership.

Examples of the consensus sequences obtained using the above-described method are illustrated in Table B 1. Examples of the exemplar sequences are shown in Table B2. Each consensus or exemplar sequence has a respective SEQ ID NO and a header that includes the qualifier (starting with “wyeCanine 1a”) and other information of that sequence. The consensus and exemplar sequences are collectively referred to as the “parent sequences.”

Table D illustrates the source(s) from which each parent sequence is derived. The source(s) for each parent sequence is represented by a pattern value (“Value”) in the formula of “XYZ.” X, Y, and Z represent the three input sequence sources, i.e., the osteoarthritis-free Canis familiaris cartilage cDNA library (“Nor.”), the osteoarthritis-affected Canis familiaris cartilage cDNA library (“Aff.”), and GenBank (“Gen.”), respectively. Each digit in “XYZ” can be either 1 or 0, depending on whether or not an input sequence for the parent sequence is derived from the source represented by that digit. For instance, if at least one input sequence is derived from the osteoarthritis-free cartilage library (“Nor.”), then the digit at “X” is selected as 1. Otherwise, “X” is 0. Similarly, if at least one input sequence is derived from the osteoarthritic cartilage cDNA library (“Aff.”) or GenBank (“Gen.”), then the digit at “Y” or “Z” is selected as 1, respectively. Otherwise, the digit at “Y” or “Z” is 0.

In one specific example, all of the input sequences for a parent sequence are derived from the osteoarthritis-free cartilage library (“Normal”). Therefore, the pattern value of that parent sequence is 100 (i.e., XYZ=100). In another specific example, all of the input sequences for a parent sequence are derived from the osteoarthritic cartilage cDNA library (“Affected”). Therefore, the pattern value of that parent sequence is 010 (i.e., XYZ=010). In yet another specific example, all of the input sequences for a parent sequence are derived from GenBank. Therefore, the pattern value of that parent sequence is 001 (i.e., XYZ=001).

Table D indicates that some of the consensus and exemplar sequences have a pattern value of 010. These sequences were collected from the 3′ sequence reads of the osteoarthritic cartilage library, but not detected in the osteoarthritis-free cartilage library. As discussed above, the chance for a sequence being detected in a cDNA library generally correlates with the level of the corresponding mRNA in the tissue from which the library is derived. Thus, the parent sequences having a pattern value of 010 can correspond to the mRNA transcripts whose levels are higher in the osteoarthritic cartilage tissue than in the non-osteoarthritic cartilage tissue. Similarly, the parent sequences having a pattern value of 011 can represent genes whose levels of expression are higher in the osteoarthritic cartilage tissue than in the non-osteoarthritic cartilage tissue.

In one embodiment, the parent sequences having a pattern value of 010 correspond to the mRNA transcripts whose levels are substantially higher in the osteoarthritic cartilage tissues than in the non-osteoarthritic cartilage tissues. For instance, the level of each of these RNA transcripts in the osteoarthritic cartilage tissues can be at least 1.5-fold, 2-fold, 3-fold, 4-fold, 5-fold, or more of that in the non-osteoarthritic cartilage tissues. The levels of mRNA transcripts can be determined using methods known in the art, such as RT-PCR, Northern Blot, or microarrays.

Table D also shows that some of the consensus and exemplar sequences have a pattern value of 100. These sequences were collected from the 3′ sequence reads of the osteoarthritis-free cartilage library, but not detected in the osteoarthritic cartilage library. Therefore, these sequences can correspond to the mRNA transcripts whose levels are substantially higher in the non-osteoarthritic cartilage tissue than in the osteoarthritic cartilage tissue. Similarly, the parent sequences having a pattern value of 101 can represent genes whose levels of expression are higher in the non-osteoarthritic cartilage tissue than in the osteoarthritic cartilage tissue.

In addition, Table D shows consensus and exemplar sequences having a pattern value of 110 or 111. These sequences are detectable in both the non-osteoarthritic and osteoarthritic cartilage tissues. The expression levels of these sequences in non-osteoarthritic cartilage tissues can be substantially the same as those in osteoarthritic cartilage tissues.

As appreciated by one of ordinary skill in the art, RNA transcripts, cDNA sequences, and other expressible sequences can be similarly collected from other animal disease models. Consensus and exemplar sequences can be generated from these sequences using the methods described above.

B. Preparation of Polynucleotide Probes for Detecting Gene Expression in Canis familiaris

The consensus and exemplar sequences depicted in Tables B1 and B2 can be used to prepare polynucleotide probes for detecting gene expression in Canis familiaris. The polynucleotide probes for each parent sequence can hybridize under stringent and/or nucleic acid array hybridization conditions to that parent sequence, or the complement thereof. Preferably, the probes for each parent sequence are incapable of hybridizing under stringent and/or nucleic acid array hybridization conditions to other parent sequences, or the complements thereof. If a parent sequence contains one or more ambiguous residues, the probes for that parent sequence can hybridize under stringent and/or nucleic acid array hybridization conditions to the longest unambiguous segment of that parent sequence. In one embodiment, the probe for a parent sequence comprises or consists of an unambiguous sequence fragment of that parent sequence, or the complement thereof.

The length of each polynucleotide probe can be selected to produce the desired hybridization effects. For example, the probes can include or consist of at least 10, 15, 20, 25, 30, 35, 40, 45, 50, 60, 70, 80, 90, 100, 200, 300, 400 or more consecutive nucleotides. The probes can be DNA, RNA, or PNA. Other modified forms of DNA, RNA, or PNA can also be used. The nucleotide units in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

In one embodiment, the probes have relatively high sequence complexity, and preferably do not contain long stretches of the same nucleotide. In another embodiment, the probes can be designed such that they do not have a high proportion of G or C residues at the 3′ ends. In yet another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. Preferably, each probe does not contain any ambiguous base.

Any part of a parent sequence can be used to prepare probes. For instance, probes can be prepared from the protein-coding region, the 5′ untranslated region, or the 3′ untranslated region of a parent sequence. Multiple probes, such as 5, 10, 15, 20, 25, 30, 40, 50, 100, 150, or more, can be prepared for each parent sequence. The multiple probes for the same parent sequence may or may not overlap each other, although overlap among different probes may be desirable in some assays.

In a preferred embodiment, the probes for a parent sequence have low sequence identities with other parent sequences, or the complements thereof. For instance, each probe for a parent sequence can have no more than 70%, 60%, 50% or less sequence identity with other parent sequences, or the complements thereof. This reduces the risk of potential cross-hybridization between the probes and the undesirable RNA transcripts. Sequence identity can be determined using methods known in the art. These methods include, but are not limited to, BLASTN, FASTA, FASTDB, and the GCG program.

The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the Genetics Computer Group (GCG).

The polynucleotide probes of the present invention can be synthesized using methods known in the art. Exemplary methods include automated or high throughput DNA synthesizers, such as those provided by Millipore, GeneMachines, and BioAutomation. Preferably, the synthesized probes are substantially free of impurities, such as incomplete products produced during the synthesis. In addition, the probes are substantially free of other contaminants that may hinder the desired functions of the probes. The probes can be purified or concentrated using different methods, such as reverse phase chromatography, ethanol precipitation, gel filtration, electrophoresis, or any combination thereof.

In one embodiment, the parent sequences with large sizes are divided into shorter sequence segments to facilitate the probe design. In addition, where a parent sequence contains both introns and exons, the intron sequences are removed such that the adjacent exons can be joined. In some cases, additional exons of the same gene are added to the exons in the original parent sequence. These divided, spliced or otherwise modified parent sequences are collectively referred to as the “tiling sequences.”

Table C depicts the tiling sequences and their respective headers. The headers include the qualifiers (starting with “wyeCanine1a:”) and other information of the tiling sequences. The first 2,676 tiling sequences in Table C correspond to, in consecutive order, the consensus sequences in Table B 1. The remaining tiling sequences correspond to, in consecutive order, the exemplar sequences in Table B2.

Table E shows the location of each tiling sequence in the corresponding parent sequence. The 5′ end of each tiling sequence in the corresponding parent sequence is indicated under “TilingStart,” and the 3′ end of the tiling sequence is shown under “TilingEnd.” For the tiling sequences that are derived by joining exons, the 5′ and 3′ ends of each of the joined exons are shown in Table E. See, for example, tiling sequences “wyeCanine1a:AJ238150.1_s_at,” “wyeCanine1a:AJ251207.1_at,” and “wyeCanine1a:AJ271090.1_at” in Table E. The sources for the additional exons that are added to the original parent sequence are also indicated in Table E. See, for example, tiling sequences “wyeCanine1a:AJ278005.1_s_at” and “wyeCanine1a:AJ302726.1_at” in Table E.

Polynucleotide probes for each tiling sequence can hybridize under stringent and/or nucleic acid array hybridization conditions to that tiling sequence, or the complement thereof. Preferably, a probe for a tiling sequence can hybridize under highly stringent conditions to the tiling sequence, or the complement thereof. More preferably, the probes for a tiling sequence are incapable of hybridizing under stringent and/or nucleic acid array hybridization conditions to other tiling sequences, or the complements thereof If a tiling sequence contains one or more ambiguous residues, the probes for the tiling sequence can hybridize under stringent and/or nucleic acid array hybridization conditions to the longest unambiguous segment of that sequence, or the complement thereof.

Any of the above-described methods can be used to prepare probes for the tiling sequences. In one embodiment, the probes are generated using Array Designer, a software package provided by TeleChem International, Inc (Sunnyvale, Calif. 94089). Examples of the probes thus generated are illustrated in Table F. The location of the 5′ and 3′ ends of each probe in the corresponding tiling sequence is shown under “5′ End” and “3′ End,” respectively. Other methods or software programs can also be used to generate hybridization probes for the tiling sequences.

The parent sequences, tiling sequences, and polynucleotide probes of the present invention can be used to detect or monitor gene expressions in any Canis familiars tissues. Methods suitable for this purpose include, but are not limited to, nucleic acid arrays (including bead arrays), Southern Blot, Northern Blot, PCR, and RT-PCR. Exemplary Canis familiaris tissues include cartilage, heart, liver, kidney, brain, lung, blood, muscle, and bone marrow.

As appreciated by those skilled in the art, polynucleotide probes suitable for detecting or monitoring gene expressions in other animal disease models can be similarly prepared using the methods described above.

C. Nucleic Acid Arrays For Detecting Gene Expression in Canis familiars

The polynucleotide probes of the present invention can be used to make nucleic acid arrays. A typical nucleic acid array includes at least one substrate support. The substrate support includes a plurality of discrete regions. The location of each discrete region is either known or determinable. The discrete regions can be organized in various forms or patterns. For instance, the discrete regions can be arranged as an array of regularly spaced areas on the surface of the substrate. Other patterns, such as linear, concentric or spiral patterns, can be used. In one embodiment, a nucleic acid array of the present invention is a bead array which includes a plurality of beads stably associated with the polynucleotide probes of the present invention.

Polynucleotide probes can be stably attached to their respective discrete regions through covalent and/or non-covalent interactions. By “stably attached,” it means that during nucleic acid array hybridization the polynucleotide probe maintains its position relative to the discrete region to which the probe is attached. Any suitable method can be used to attach polynucleotide probes to a nucleic acid array substrate. In one embodiment, the attachment is achieved by first depositing the polynucleotide probes to their respective discrete regions and then exposing the surface to a solution of a cross-linking agent, such as glutaraldehyde, borohydride, or other bifinctional agents. In another embodiment, the polynucleotide probes are covalently bound to the substrate via an alkylamino-linker group or by coating the glass slides with polyethylenimine followed by activation with cyanuric chloride for coupling the polynucleotides. In yet another embodiment, the polynucleotide probes are covalently attached to a nucleic acid array through polymer linkers. The polymer linkers may improve the accessibility of the probes to their purported targets. Preferably, the polymer linkers are not involved in the interactions between the probes and their purported targets.

In addition, the polynucleotide probes can be stably attached to a nucleic acid array substrate through non-covalent interactions. In one embodiment, the polynucleotide probes are attached to the substrate through electrostatic interactions between positively charged surface groups and the negatively charged probes. In another embodiment, the substrate is a glass slide having a coating of a polycationic polymer on its surface, such as a cationic polypeptide. The probes are bound to these polycationic polymers. In yet another embodiment, the methods described in U.S. Pat. No. 6,440,723, which is incorporated herein by reference, are used to attach the probes to the nucleic acid array substrate(s).

Various materials can be used to make the substrate support. Suitable materials include, but are not limited to, glasses, silica, ceramics, nylons, quartz wafers, gels, metals, and papers. The substrates can be flexible or rigid. In one embodiment, they are in the form of a tape that is wound up on a reel or cassette. Two or more substrate supports can be used in the same nucleic acid array. Preferably, the substrate is non-reactive with reagents that are used in nucleic acid array hybridization.

The surfaces of the substrate support can be smooth and substantially planar. The surfaces of the substrate can also have a variety of configurations, such as raised or depressed regions, trenches, v-grooves, mesa structures, and other irregularities. The surfaces of the substrate can be coated with one or more modification layers. Suitable modification layers include inorganic and organic layers, such as metals, metal oxides, polymers, or small organic molecules. In one embodiment, the surface(s) of the substrate is chemically treated to include groups such as hydroxyl, carboxyl, amine, aldehyde, or sulfhydryl groups.

The discrete regions on the substrate can be of any size, shape and density. For instance, they can be squares, ellipsoids, rectangles, triangles, circles, other regular geometric or irregular geometric shapes, or any portion or combination thereof. In one embodiment, each of the discrete regions has a surface area of less than 10⁻¹ cm², such as less than 10⁻², 10⁻³, 10⁻⁴, 10⁻⁶, or 10⁻⁷ cm². In another embodiment, the discrete region and its closest neighbor, measured from center-to-center, is in the range of from about 10 to about 400 μm. The density of the discrete regions may range, for example, between 50 and 50,000 regions/cm².

All of the methods known in the art can be used to make the nucleic acid arrays of the present invention. For instance, the probes can be synthesized in a step-by-step manner on the substrate, or can be attached to the substrate in pre-synthesized forms. Algorithms for reducing the number of synthesis cycles can be used. In one embodiment, a nucleic acid array of the present invention is synthesized in a combinational fashion by delivering monomers to the discrete regions through mechanically constrained flowpaths. In another embodiment, a nucleic acid array of the present invention is synthesized by spotting monomer reagents onto a substrate support using an ink jet printer (such as the DeskWriter C manufactured by Hewlett-Packard). In yet another embodiment, polynucleotide probes are immobilized on a nucleic acid array of the present invention by using photolithography techniques.

In one embodiment, a nucleic acid array of the present invention comprises one or more polynucleotide probes, each of which is capable of hybridizing under stringent and/or nucleic acid array hybridization conditions to a different respective mRNA transcript, or the complement thereof. The frequency of occurrence of each of these mRNA transcripts is substantially higher in osteoarthritic cartilage tissues than in non-osteoarthritic cartilage tissues. The osteoarthritic and non-osteoarthritic cartilage tissues can be derived from the same animal or from different animals. Suitable animals include dogs, rabbits, rats, mice, hamsters, guinea pigs, or any other animal that may be used as a model of osteoarthritis.

Any number of the polynucleotide probes can be included in the nucleic acid arrays of the present invention for detecting the differentially expressed mRNA transcripts. In one embodiment, a nucleic acid array of the present invention includes at least 2, 5, 10, 20, 30, 40, 50, 100, 200, 300, 400, 500, 1,000, or more different probes, and each probe can hybridize under stringent and/or nucleic acid array hybridization conditions to a different respective mRNA transcript. The level of expression of each of these mRNA transcripts is substantially higher in the osteoarthritic cartilage tissues than in the non-osteoarthritic cartilage tissues. For instance, the level of expression of each of these mRNA transcripts in the osteoarthritic cartilage tissues can be at least 1.5-fold, 2-fold, 3-fold, 4-fold, 5-fold, 10-fold, or more of that in the non-osteoarthritic cartilage tissues. Suitable polynucleotide probes for this embodiment can be selected from Table F. These probes can hybridize under stringent and/or nucleic acid array hybridization conditions to the tiling sequences with a pattern value of 010 or 011.

In another embodiment, a nucleic acid array of the present invention further comprises one or more polynucleotide probes, each of which is capable of hybridizing under stringent and/or nucleic acid array hybridization conditions to a different respective mRNA transcript, or the complement thereof, where the level of expression of each of the respective mRNA transcripts is substantially higher in the non-osteoarthritic cartilage tissues than in the osteoarthritic cartilage tissues. For instance, the level of expression of each of these mRNA transcripts in the non-osteoarthritic cartilage tissues can be at least 1.5-fold, 2-fold, 3-fold, 4-fold, 5-fold, 10-fold, or more of that in the osteoarthritic cartilage tissues. The nucleic acid array may include at least 2, 5, 10, 20, 30, 40, 50, 100, 200, 300, 400, 500, 1,000, or more probes of this type. Suitable probes for this embodiment can be selected from Table F. These probes can hybridize under stringent and/or nucleic acid array hybridization conditions to the tiling sequences with a pattern value of 100 or 101. Probes for the tiling sequences with other pattern values can also be included in the nucleic acid array.

In yet another embodiment, a nucleic acid array of the present invention comprises a plurality of polynucleotide probes, each of which can hybridize under stringent and/or nucleic acid array hybridization conditions to a different respective tiling sequence selected from Table C, or the complement thereof. The tiling sequences can have any pattern value, such as 001, 010, 011, 100, 101, 110, or 111. The plurality of polynucleotide probes can include at least 2, 5, 10, 20, 30, 40, 50, 100, 200, 300, 400, 500, 1,000, 5,000, 10,000, or more different probes. Suitable probes for this embodiment can be selected from Table F.

In still yet another embodiment, a nucleic acid array of the present invention comprises at least one probe for each tiling sequence selected from Table C. Preferably, the nucleic acid array includes two or more probes (such as 4, 6, 8, 10, 12, 14, 16, 18, 20, 30, 40, 50, 60, or more) for each tiling sequence selected from Table C. In a further embodiment, a nucleic acid array of the present invention includes each and every oligonucleotide probe selected from Table F.

The length of each probe on a nucleic acid array of the present invention can be selected to achieve the desirable hybridization effects. For instance, each probe can include or consist of 15, 20, 25, 30, 35, 40, 45, 50, 60, 70, 80, 90, 100 or more consecutive nucleotides. In one embodiment, each probe consists of 25 consecutive nucleotides.

Different probes can be included in a nucleic acid array for detecting the same tiling sequence selected from Table C. For instance, at least 2, 5, 10, 15, 20, 25, 30 or more different probes can be used for detecting the same tiling sequence. In one specific example, 16 different probes are used for detecting the same tiling sequence. In another specific examples, at least 12 different probes are used for detecting the same tiling sequence. Each of these different probes can be attached to a different respective discrete region on a nucleic acid array. Alternatively, two or more different probes can be attached to the same discrete region. The concentration of one probe with respect to the other probe or probes in the same region may vary according to the objectives and requirements of the particular experiment. In one embodiment, different probes in the same region are present in approximately equimolar ratio.

Preferably, probes for different tiling sequences are attached to different discrete regions on a nucleic acid array. In some applications, probes for different tiling sequences are attached to the same discrete region.

A nucleic acid array of the present invention can further include a plurality of control probes which can hybridize under stringent and/or nucleic acid array hybridization conditions to the respective control sequences, or the complements thereof. Examples of control sequences suitable for the present invention are listed in Table G. Like the parent sequences, each ontrol sequence in Table G has a SEQ ID NO and a header that includes the qualifier (starting with “wyeCanine 1a”) and other information of the control sequence.

TABLE G
Control Sequences (SEQ ID NOS: 12,168-12,311)

| SEQ ID | Header |
|---|---|
| 12168 | >control:wyeCanine1a:18SRNA3_Hs_at; Unassigned; Human |
| | 18S rRNA gene, complete. |
| 12169 | >control:wyeCanine1a:18SRNA3_Mm_at; Unassigned; Mouse |
| | gene for 18S rRNA. |
| 12170 | >control:wyeCanine1a:18SRNA5_Hs_at; Unassigned; Human |
| | 18S rRNA gene, complete. |
| 12171 | >control:wyeCanine1a:18SRNA5_Mm_at; Unassigned; Mouse |
| | gene for 18S rRNA. |
| 12172 | >control:wyeCanine1a:18SRNAM_Hs_at; Unassigned; Human |
| | 18S rRNA gene, complete. |
| 12173 | >control:wyeCanine1a:18SRNAM_Mm_at; Unassigned; Mouse |
| | gene for 18S rRNA. |
| 12174 | >control:wyeCanine1a:28SRNAM_Cf_at; 28SRNAM_Cf; |
| | AJ388541.1 Canis familiaris 28S rRNA gene, clone BC50 |
| 12175 | >control:wyeCanine1a:AFFX-18SRNAMur/X00686_3_at; |
| | X00686; X00686 Mouse gene for 18S rRNA (_5, _M, _3 |
| | represent transcript regions 5 prime, Middle, and 3 prime respectively) |
| 12176 | >control:wyeCanine1a:AFFX-18SRNAMur/X00686_5_at; |
| | X00686; X00686 Mouse gene for 18S rRNA (_5, _M, _3 |
| | represent transcript regions 5 prime, Middle, and 3 prime respectively) |
| 12177 | >control:wyeCanine1a:AFFX-18SRNAMur/X00686_M_at; |
| | X00686; X00686 Mouse gene for 18S rRNA (_5, _M, _3 |
| | represent transcript regions 5 prime, Middle, and 3 prime respectively) |
| 12178 | >control:wyeCanine1a:AFFX-b-ActinMur/M12481_3_at; |
| | M12481; M12481 Mouse cytoplasmic beta-actin mRNA (_5, _M, |
| | _3 represent transcript regions 5 prime, Middle, and 3 prime respectively) |
| 12179 | >control:wyeCanine1a:AFFX-b-ActinMur/M12481_5_at; |
| | M12481; M12481 Mouse cytoplasmic beta-actin mRNA (_5, _M, |
| | _3 represent transcript regions 5 prime, Middle, and 3 prime respectively) |
| 12180 | >control:wyeCanine1a:AFFX-b-ActinMur/M12481_M_at; |
| | M12481; M12481 Mouse cytoplasmic beta-actin mRNA (_5, _M, |
| | _3 represent transcript regions 5 prime, Middle, and 3 prime respectively) |
| 12181 | >control:wyeCanine1a:AFFX-BioB-3_at; J04423; J04423 E coli |
| | bioB gene biotin synthetase (-5, -M, -3 represent transcript |
| | regions 5 prime, Middle, and 3 prime respectively) |
| 12182 | >control:wyeCanine1a:AFFX-BioB-5_at; J04423; J04423 E coli |
| | bioB gene biotin synthetase (-5, -M, -3 represent transcript |
| | regions 5 prime, Middle, and 3 prime respectively) |
| 12183 | >control:wyeCanine1a:AFFX-BioB-M_at; J04423; J04423 E coli |
| | bioB gene biotin synthetase (-5, -M, -3 represent transcript |
| | regions 5 prime, Middle, and 3 prime respectively) |
| 12184 | >control:wyeCanine1a:AFFX-BioC-3_at; J04423; J04423 E coli |
| | bioC protein (-5 and -3 represent transcript regions 5 prime and 3 prime respectively) |
| 12185 | >control:wyeCanine1a:AFFX-BioC-5_at; J04423; J04423 E coli |
| | bioC protein (-5 and -3 represent transcript regions 5 prime and 3 prime respectively) |
| 12186 | >control:wyeCanine1a:AFFX-BioDn-3_at; J04423; J04423 E coli |
| | bioD gene dethiobiotin synthetase (-5 and -3 represent transcript |
| | regions 5 prime and 3 prime respectively) |
| 12187 | >control:wyeCanine1a:AFFX-BioDn-5_at; J04423; J04423 E coli |
| | bioD gene dethiobiotin synthetase (-5 and -3 represent transcript |
| | regions 5 prime and 3 prime respectively) |
| 12188 | >control:wyeCanine1a:AFFX-CreX-3_at; X03453; X03453 |
| | Bacteriophage P1 cre recombinase protein (-5 and -3 represent |
| | transcript regions 5 prime and 3 prime respectively) |
| 12189 | >control:wyeCanine1a:AFFX-CreX-5_at; X03453; X03453 |
| | Bacteriophage P1 cre recombinase protein (-5 and -3 represent |
| | transcript regions 5 prime and 3 prime respectively) |
| 12190 | >control:wyeCanine1a:AFFX-DapX-3_at; L38424; L38424 B |
| | subtilis dapB, jojF, jojG genes corresponding to nucleotides 1358-3197 |
| | of L38424 (-5, -M, -3 represent transcript regions 5 prime, |
| | Middle, and 3 prime respectively) |
| 12191 | >control:wyeCanine1a:AFFX-DapX-5_at; L38424; L38424 B |
| | subtilis dapB, jojF, jojG genes corresponding to nucleotides 1358-3197 |
| | of L38424 (-5, -M, -3 represent transcript regions 5 prime, |
| | Middle, and 3 prime respectively) |
| 12192 | >control:wyeCanine1a:AFFX-DapX-M_at; L38424; L38424 B |
| | subtilis dapB, jojF, jojG genes corresponding to nucleotides 1358-3197 |
| | of L38424 (-5, -M, -3 represent transcript regions 5 prime, |
| | Middle, and 3 prime respectively) |
| 12193 | >control:wyeCanine1a:AFFX-GapdhMur/M32599_3_at; M32599; |
| | M32599 Mouse glyceraldehyde-3-phosphate dehydrogenase |
| | mRNA, complete cds (_5, _M, _3 represent transcript regions 5 |
| | prime, Middle, and 3 prime respectively) |
| 12194 | >control:wyeCanine1a:AFFX-GapdhMur/M32599_5_at; M32599; |
| | M32599 Mouse glyceraldehyde-3-phosphate dehydrogenase |
| | mRNA, complete cds (_5, _M, _3 represent transcript regions 5 |
| | prime, Middle, and 3 prime respectively) |
| 12195 | >control:wyeCanine1a:AFFX-GapdhMur/M32599_M_at; |
| | M32599; M32599 Mouse glyceraldehyde-3-phosphate |
| | dehydrogenase mRNA, complete cds (_5, _M, _3 represent |
| | transcript regions 5 prime, Middle, and 3 prime respectively) |
| 12196 | >control:wyeCanine1a:AFFX-HSAC07/X00351_3_at; X00351; |
| | X00351 Human mRNA for beta-actin (_5, _M, _3 represent |
| | transcript regions 5 prime, Middle, and 3 prime respectively) |
| 12197 | >control:wyeCanine1a:AFFX-HSAC07/X00351_5_at; X00351; |
| | X00351 Human mRNA for beta-actin (_5, _M, _3 represent |
| | transcript regions 5 prime, Middle, and 3 prime respectively) |
| 12198 | >control:wyeCanine1a:AFFX-HSAC07/X00351_M_at; X00351; |
| | X00351 Human mRNA for beta-actin (_5, _M, _3 represent |
| | transcript regions 5 prime, Middle, and 3 prime respectively) |
| 12199 | >control:wyeCanine1a:AFFX-hum_alu_at; U14573; U14573 |
| | Human Alu-Sq subfamily consensus sequence. |
| 12200 | >control:wyeCanine1a:AFFX-HUMGAPDH/M33197_3_at; |
| | M33197; M33197 Human glyceraldehyde-3-phosphate |
| | dehydrogenase (GAPDH) mRNA, complete cds (_5, _M, _3 |
| | represent transcript regions 5 prime, Middle, and 3 prime respectively) |
| 12201 | >control:wyeCanine1a:AFFX-HUMGAPDH/M33197_5_at; |
| | M33197; M33197 Human glyceraldehyde-3-phosphate |
| | dehydrogenase (GAPDH) mRNA, complete cds (_5, _M, _3 |
| | represent transcript regions 5 prime, Middle, and 3 prime respectively) |
| 12202 | >control:wyeCanine1a:AFFX-HUMGAPDH/M33197_M_at; |
| | M33197; M33197 Human glyceraldehyde-3-phosphate |
| | dehydrogenase (GAPDH) mRNA, complete cds (_5, _M, _3 |
| | represent transcript regions 5 prime, Middle, and 3 prime respectively) |
| 12203 | >control:wyeCanine1a:AFFX-HUMISGF3A/M97935_3_at; |
| | M97935; M97935 Homo sapiens transcription factor ISGF-3 |
| | mRNA, complete cds (_5, _MA, MB, _3 represent transcript |
| | regions 5 prime, MiddleA, MiddleB, and 3 prime respectively) |
| 12204 | >control:wyeCanine1a:AFFX-HUMISGF3A/M97935_5_at; |
| | M97935; M97935 Homo sapiens transcription factor ISGF-3 |
| | mRNA, complete cds (_5, _MA, MB, _3 represent transcript |
| | regions 5 prime, MiddleA, MiddleB, and 3 prime respectively) |
| 12205 | >control:wyeCanine1a:AFFX-HUMISGF3A/M97935_MA_at; |
| | M97935; M97935 Homo sapiens transcription factor ISGF-3 |
| | mRNA, complete cds (_5, _MA, MB, _3 represent transcript |
| | regions 5 prime, MiddleA, MiddleB, and 3 prime respectively) |
| 12206 | >control:wyeCanine1a:AFFX-HUMISGF3A/M97935_MB_at; |
| | M97935; M97935 Homo sapiens transcription factor ISGF-3 |
| | mRNA, complete cds (_5, _MA, MB, _3 represent transcript |
| | regions 5 prime, MiddleA, MiddleB, and 3 prime respectively) |
| 12207 | >control:wyeCanine1a:AFFX-HUMRGE/M10098_3_at; M10098; |
| | M10098 Human 18S rRNA gene, complete (_5, _M, _3 represent |
| | transcript regions 5 prime, Middle, and 3 prime respectively) |
| 12208 | >control:wyeGanine1a:AFFX-HUMRGE/M10098_5_at; M10098; |
| | M10098 Human 18S rRNA gene, complete (_5, _M, _3 represent |
| | transcript regions 5 prime, Middle, and 3 prime respectively) |
| 12209 | >control:wyeCanine1a:AFFX-HUMRGE/M10098_M_at; |
| | M10098; M10098 Human 18S rRNA gene, complete (_5, _M, _3 |
| | represent transcript regions 5 prime, Middle, and 3 prime respectively) |
| 12210 | >control:wyeCanine1a:AFFX-LysX-3_at; X17013; X17013 B |
| | subtilis lys gene for diaminopimelate decarboxylase corresponding |
| | to nucleotides 350-1345 of X17013 (-5, -M, -3 represent |
| | transcript regions 5 prime, Middle, and 3 prime respectively) |
| 12211 | >control:wyeCanine1a:AFFX-LysX-5_at; X17013; X17013 B |
| | subtilis lys gene for diaminopimelate decarboxylase corresponding |
| | to nucleotides 350-1345 of X17013 (-5, -M, -3 represent |
| | transcript regions 5 prime, Middle, and 3 prime respectively) |
| 12212 | >control:wyeCanine1a:AFFX-LysX-M_at; X17013; X17013 B |
| | subtilis lys gene for diaminopimelate decarboxylase corresponding |
| | to nucleotides 350-1345 of X17013 (-5, -M, -3 represent |
| | transcript regions 5 prime, Middle, and 3 prime respectively) |
| 12213 | >control:wyeCanine1a:AFFX-M27830_3_at; M27830; M27830 |
| | Human 28S ribosomal RNA gene, complete cds (_5, _M, _3 |
| | represent transcript regions 5 prime, Middle, and 3 prime respectively) |
| 12214 | >control:wyeCanine1a:AFFX-M27830_5_at; M27830; M27830 |
| | Human 28S ribosomal RNA gene, complete cds (_5, _M, _3 |
| | represent transcript regions 5 prime, Middle, and 3 prime respectively) |
| 12215 | >control:wyeCanine1a:AFFX-M27830_M_at; M27830; M27830 |
| | Human 28S ribosomal RNA gene, complete cds (_5, _M, _3 |
| | represent transcript regions 5 prime, Middle, and 3 prime respectively) |
| 12216 | >control:wyeCanine1a:AFFX-MUR_b2_at; X63136; X63136 |
| | M. musculus DNA for intragenic sequence including B2 element |
| 12217 | >control:wyeCanine1a:AFFX-MURINE_b1_at; U01310; U01310 |
| | Mus musculus C57/Black6 BC1 scRNA |
| 12218 | >control:wyeCanine1a:AFFX-MURINE_B2_at; K00131; K00131 |
| | mouse b2 repeat sequence from clone mm61 |
| 12219 | >control:wyeCanine1a:AFFX-PheX-3_at; M24537; M24537 B |
| | subtilis pheB, pheA genes corresponding to nucleotides 2017-3334 |
| | of M24537 (-5, -M, -3 represent transcript regions 5 prime, Middle, and 3 |
| | prime respectively) |
| 12220 | >control:wyeCanine1a:AFFX-PheX-5_at; M24537; M24537 B |
| | subtilis pheB, pheA genes corresponding to nucleotides 2017-3334 |
| | of M24537 (-5, -M, -3 represent transcript regions 5 prime, Middle, and 3 |
| | prime respectively) |
| 12221 | >control:wyeCanine1a:AFFX-PheX-M_at; M24537; M24537 B |
| | subtilis pheB, pheA genes corresponding to nucleotides 2017-3334 |
| | of M24537 (-5, -M, -3 represent transcript regions 5 prime, Middle, and 3 |
| | prime respectively) |
| 12222 | >control:wyeCanine1a:AFFX-PyruCarbMur/L09192_3_at; |
| | L09192; L09192 Mus musculus pyruvate carboxylase mRNA, |
| | complete cds (_5, _MA, MB, _3 represent transcript regions 5 |
| | prime, MiddleA, MiddleB, and 3 prime respectively) |
| 12223 | >control:wyeCanine1a:AFFX-PyruCarbMur/L09192_5_at; |
| | L09192; L09192 Mus musculus pyruvate carboxylase mRNA, |
| | complete cds (_5, _MA, MB, _3 represent transcript regions 5 |
| | prime, MiddleA, MiddleB, and 3 prime respectively) |
| 12224 | >control:wyeCanine1a:AFFX-PyruCarbMur/L09192_MA_at; |
| | L09192; L09192 Mus musculus pyruvate carboxylase mRNA, |
| | complete cds (_5, _MA, MB, _3 represent transcript regions 5 |
| | prime, MiddleA, MiddleB, and 3 prime respectively) |
| 12225 | >control:wyeCanine1a:AFFX-PyruCarbMur/L09192_MB_at; |
| | L09192; L09192 Mus musculus pyruvate carboxylase mRNA, |
| | complete cds (_5, _MA, MB, _3 represent transcript regions 5 |
| | prime, MiddleA, MiddleB, and 3 prime respectively) |
| 12226 | >control:wyeCanine1a:AFFX-r2-Bs-dap-3_at; L38424; Bacillus |
| | subtilis /REF = L38424 /DEF = B subtilis dapB, jojF, jojG genes |
| | corresponding to nucleotides 2634-3089 of L38424 /LEN = 1931 (-5, |
| | -M, -3 represent transcript regions 5 prime, Middle, and 3 prime respectively) |
| 12227 | >control:wyeCanine1a:AFFX-r2-Bs-dap-5_at; L38424; Bacillus |
| | subtilis /REF = L38424 /DEF = B subtilis dapB, jojF, jojG genes |
| | corresponding to nucleotides 1439-1846 of L38424 /LEN = 1931 (-5, |
| | -M, -3 represent transcript regions 5 prime, Middle, and 3 prime respectively) |
| 12228 | >control:wyeCanine1a:AFFX-r2-Bs-dap-M_at; L38424; Bacillus |
| | subtilis /REF = L38424 /DEF = B subtilis dapB, jojF, jojG genes |
| | corresponding to nucleotides 2055-2578 of L38424 /LEN = 1931 (-5, |
| | -M, -3 represent transcript regions 5 prime, Middle, and 3 prime respectively) |
| 12229 | >control:wyeCanine1a:AFFX-r2-Bs-lys-3_at; X17013; Bacillus |
| | subtilis /REF = X17013 /DEF = B subtilis lys gene for |
| | diaminopimelate decarboxylase corresponding to nucleotides |
| | 1008-1263 of X17013 /LEN = 1108 (-5, -M, -3 represent transcript |
| | regions 5 prime, Middle, and 3 prime respectively) |
| 12230 | >control:wyeCanine1a:AFFX-r2-Bs-lys-5_at; X17013; Bacillus |
| | subtilis /REF = X17013 /DEF = B subtilis lys gene for |
| | diaminopimelate decarboxylase corresponding to nucleotides 411-659 |
| | of X17013 /LEN = 1108 (-5, -M, -3 represent transcript regions |
| | 5 prime, Middle, and 3 prime respectively) |
| 12231 | >control:wyeCanine1a:AFFX-r2-Bs-lys-M_at; X17013; Bacillus |
| | subtilis /REF = X17013 /DEF = B subtilis lys gene for |
| | diaminopimelate decarboxylase corresponding to nucleotides 673-1002 |
| | of X17013 /LEN = 1108 (-5, -M, -3 represent transcript |
| | regions 5 prime, Middle, and 3 prime respectively) |
| 12232 | >control:wyeCanine1a:AFFX-r2-Bs-phe-3_at; M24537; Bacillus |
| | subtilis /REF = M24537 /DEF = B subtilis pheB, pheA genes |
| | corresponding to nucleotides 2897-3200 of M24537 /LEN = 1409 (-5, |
| | -M, -3 represent transcript regions 5 prime, Middle, and 3 prime respectively) |
| 12233 | >control:wyeCanine1a:AFFX-r2-Bs-phe-5_at; M24537; Bacillus |
| | subtilis /REF = M24537 /DEF = B subtilis pheB, pheA genes |
| | corresponding to nucleotides 2116-2382 of M24537 /LEN = 1409 (-5, |
| | -M, -3 represent transcript regions 5 prime, Middle, and 3 prime respectively) |
| 12234 | >control:wyeCanine1a:AFFX-r2-Bs-phe-M_at; M24537; Bacillus |
| | subtilis /REF= M24537 /DEF = B subtilis pheB, pheA genes |
| | corresponding to nucleotides 2484-2875 of M24537 /LEN = 1409 (-5, |
| | -M, -3 represent transcript regions 5 prime, Middle, and 3 prime respectively) |
| 12235 | >control:wyeCanine1a:AFFX-r2-Bs-thr-3_s_at; X04603; Bacillus |
| | subtilis /REF= X04603 /DEF = B subtilis thrC, thrB genes |
| | corresponding to nucleotides 1689-2151 of X04603 /LEN = 2073 (-5, |
| | -M, -3 represent transcript regions 5 prime, Middle, and 3 prime respectively) |
| 12236 | >control:wyeCanine1a:AFFX-r2-Bs-thr-5_s_at; X04603; Bacillus |
| | subtilis /REF = X04603 /DEF = B subtilis thrC, thrB genes |
| | corresponding to nucleotides 288-932 of X04603 /LEN = 2073 (-5, |
| | -M, -3 represent transcript regions 5 prime, Middle, and 3 prime respectively) |
| 12237 | >control:wyeCanine1a:AFFX-r2-Bs-thr-M_s_at; X04603; Bacillus |
| | subtilis /REF = X04603 /DEF = B subtilis thrC, thrB genes |
| | corresponding to nucleotides 995-1562 of X04603 /LEN = 2073 (-5, |
| | -M, -3 represent transcript regions 5 prime, Middle, and 3 prime respectively) |
| 12238 | >control:wyeCanine1a:AFFX-r2-Ec-bioB-3_at; J04423; |
| | Escherichia coli /REF = J04423 /DEF = E coli bioB gene biotin |
| | synthetase corresponding to nucleotides 2772-3004 of J04423 |
| | /LEN = 1114 (-5, -M, -3 represent transcript regions 5 prime, |
| | Middle, and 3 prime respectively) |
| 12239 | >control:wyeCanine1a:AFFX-r2-Ec-bioB-5_at; J04423; |
| | Escherichia coli /REF = J04423 /DEF = E coli bioB gene biotin |
| | synthetase corresponding to nucleotides 2071-2304 of J04423 |
| | /LEN = 1114 (-5, -M, -3 represent transcript regions 5 prime, |
| | Middle, and 3 prime respectively) |
| 12240 | >control:wyeCanine1a:AFFX-r2-Ec-bioB-M_at; J04423; |
| | Escherichia coli /REF = J04423 /DEF = E coli bioB gene biotin |
| | synthetase corresponding to nucleotides 2393-2682 of J04423 |
| | /LEN = 1114 (-5, -M, -3 represent transcript regions 5 prime, |
| | Middle, and 3 prime respectively) |
| 12241 | >control:wyeCanine1a:AFFX-r2-Ec-bioC-3_at; J04423; |
| | Escherichia coli /REF = J04423 /DEF = E coli bioC protein |
| | corresponding to nucleotides 4609-4883 of J04423 /LEN = 777 (-5 |
| | and -3 represent transcript regions 5 prime and 3 prime respectively) |
| 12242 | >control:wyeCanine1a:AFFX-r2-Ec-bioC-5_at; J04423; |
| | Escherichia coli /REF = J04423 /DEF = E coli bioC protein |
| | corresponding to nucleotides 4257-4573 of J04423 /LEN = 777 (-5 |
| | and -3 represent transcript regions 5 prime and 3 prime respectively) |
| 12243 | >control:wyeCanine1a:AFFX-r2-Ec-bioD-3_at; J04423; |
| | Escherichia coli /REF = J04423 /DEF = E coli bioD gene |
| | dethiobiotin synthetase corresponding to nucleotides 5312-5559 of |
| | J04423 /LEN = 676 (-5 and -3 represent transcript regions 5 prime |
| | and 3 prime respectively) |
| 12244 | >control:wyeCanine1a:AFFX-r2-Ec-bioD-5_at; J04423; |
| | Escherichia coli /REF = J04423 /DEF = E coli bioD gene |
| | dethiobiotin synthetase corresponding to nucleotides 5024-5244 of |
| | J04423 /LEN = 676 (-5 and -3 represent transcript regions 5 prime |
| | and 3 prime respectively) |
| 12245 | >control:wyeCanine1a:AFFX-r2-P1-cre-3_at; X03453; |
| | Bacteriophage /REF = X03453 /DEF = Bacteriophage P1 cre |
| | recombinase protein corresponding to nucleotides 1032-1270 of |
| | X03453 /LEN = 1058 (-5 and -3 represent transcript regions 5 |
| | prime and 3 prime respectively) |
| 12246 | >control:wyeCanine1a:AFFX-r2-P1-cre-5_at; X03453; |
| | Bacteriophage /REF = X03453 /DEF = Bacteriophage P1 cre |
| | recombinase protein corresponding to nucleotides 581-1001 of |
| | X03453 /LEN = 1058 (-5 and -3 represent transcript regions 5 |
| | prime and 3 prime respectively) |
| 12247 | >control:wyeCanine1a:AFFX-ThrX-3_at; X04603; X04603 B |
| | subtilis thrC, thrB genes corresponding to nucleotides 248-2229 of |
| | X04603 (-5, -M, -3 represent transcript regions 5 prime, Middle, |
| | and 3 prime respectively) |
| 12248 | >control:wyeCanine1a:AFFX-ThrX-5_at; X04603; X04603 B |
| | subtilis thrC, thrB genes corresponding to nucleotides 248-2229 of |
| | X04603 (-5, -M, -3 represent transcript regions 5 prime, Middle, |
| | and 3 prime respectively) |
| 12249 | >control:wyeCanine1a:AFFX-ThrX-M_at; X04603; X04603 B |
| | subtilis thrC, thrB genes corresponding to nucleotides 248-2229 of |
| | X04603 (-5, -M, -3 represent transcript regions 5 prime, Middle, |
| | and 3 prime respectively) |
| 12250 | >control:wyeCanine1a:AFFX-TransRecMur/X57349_3_at; |
| | X57349; X57349 M. musculus mRNA for transferrin receptor (_5, |
| | _M, _3 represent transcript regions 5 prime, Middle, and 3 prime |
| | respectively) |
| 12251 | >control:wyeCanine1a:AFFX-TransRecMur/X57349_5_at; |
| | X57349; X57349 M. musculus mRNA for transferrin receptor (_5, |
| | _M, _3 represent transcript regions 5 prime, Middle, and 3 prime |
| | respectively) |
| 12252 | >control:wyeCanine1a:AFFX-TransRecMur/X57349_M_at; |
| | X57349; X57349 M. musculus mRNA for transferrin receptor (_5, |
| | _M, _3 represent transcript regions 5 prime, Middle, and 3 prime |
| | respectively) |
| 12253 | >control:wyeCanine1a:AFFX-TrpnX-3_at; K01391; K01391 B |
| | subtilis TrpE protein, TrpD protein, TrpC protein corresponding to |
| | nucleotides 1883-4400 of K01391 (-5, -M, -3 represent transcript |
| | regions 5 prime, Middle, and 3 prime respectively) |
| 12254 | >control:wyeCanine1a:AFFX-TrpnX-5_at; K01391; K01391 B |
| | subtilis TrpE protein, TrpD protein, TrpC protein corresponding to |
| | nucleotides 1883-4400 of K01391 (-5, -M, -3 represent transcript |
| | regions 5 prime, Middle, and 3 prime respectively) |
| 12255 | >control:wyeCanine1a:AFFX-TrpnX-M_at; K01391; K01391 B |
| | subtilis TrpE protein, TrpD protein, TrpC protein corresponding to |
| | nucleotides 1883-4400 of K01391 (-5, -M, -3 represent transcript |
| | regions 5 prime, Middle, and 3 prime respectively) |
| 12256 | >control:wyeCanine1a:BACTIN3_Cf_at; BACTIN3_Cf; Cluster |
| | includes AF021873.2 Canis familiaris beta-actin mRNA, complete cds. |
| 12257 | >control:wyeCanine1a:BACTIN3_Hs_at; Unassigned; Human |
| | mRNA for beta-actin. |
| 12258 | >control:wyeCanine1a:bACTIN3_Mm_at; Unassigned; Mouse |
| | cytoplasmic beta-actin mRNA. |
| 12259 | >control:wyeCanine1a:BACTIN5_Cf_at; BACTIN5_Cf; Cluster |
| | includes AF021873.2 Canis familiaris beta-actin mRNA, complete cds. |
| 12260 | >control:wyeCanine1a:BACTIN5_Hs_at; Unassigned; Human |
| | mRNA for beta-actin. |
| 12261 | >control:wyeCanine1a:bACTIN5_Mm_at; Unassigned; Mouse |
| | cytoplasmic beta-actin mRNA. |
| 12262 | >control:wyeCanine1a:BACTINM_Cf_at; BACTINM_Cf; Cluster |
| | includes AF021873.2 Canis familiaris beta-actin mRNA, complete cds. |
| 12263 | >control:wyeCanine1a:BACTINM_Hs_at; Unassigned; Human |
| | mRNA for beta-actin. |
| 12264 | >control:wyeCanine1a:bACTINM_Mm_at; Unassigned; Mouse |
| | cytoplasmic beta-actin mRNA. |
| 12265 | >control:wyeCanine1a:BIOB3_at; Unassigned; E. coli biotin |
| | synthetase (bioB), complete cds. |
| 12266 | >control:wyeCanine1a:BIOB5_at; Unassigned; E. coli biotin |
| | synthetase (bioB), complete cds. |
| 12267 | >control:wyeCanine1a:BIOBM_at; Unassigned; E. coli biotin |
| | synthetase (bioB), complete cds. |
| 12268 | >control:wyeCanine1a:BIOC3_at; Unassigned; E. coli bioC |
| | protein, complete cds. |
| 12269 | >control:wyeCanine1a:BIOC5_at; Unassigned; E. coli bioC |
| | protein, complete cds. |
| 12270 | >control:wyeCanine1a:BIOD3_at; Unassigned; E. coli dethiobiotin |
| | synthetase (bioD), complete cds. |
| 12271 | >control:wyeCanine1a:BIOD5_at; Unassigned; E. coli dethiobiotin |
| | synthetase (bioD), complete cds. |
| 12272 | >control:wyeCanine1a:CRE3_at; Unassigned; Bacteriophage P1 |
| | cre gene for recombinase protein. |
| 12273 | >control:wyeCanine1a:CRE5_at; Unassigned; Bacteriophage P1 |
| | cre gene for recombinase protein. |
| 12274 | >control:wyeCanine1a:DAP3_at; Unassigned; Bacillus subtilis |
| | dihydropicolinate reductase (dapB), jojF, jojG, complete cds's. |
| 12275 | >control:wyeCanine1a:DAP5_at; Unassigned; Bacillus subtilis |
| | dihydropicolinate reductase (dapB), jojF, jojG, complete cds's. |
| 12276 | >control:wyeCanine1a:DAPM_at; Unassigned; Bacillus subtilis |
| | dihydropicolinate reductase (dapB), jojF, jojG, complete cds's. |
| 12277 | >control:wyeCanine1a:GAPDH3_Cf_x_at; GAPDH3_Cf; Cluster |
| | includes AB038240.1 Canis familiaris GAPDH mRNA for |
| | glyceraldehyde-3-phosphate dehydrogenase, complete cds. |
| 12278 | >control:wyeCanine1a:GAPDH3_Hs_at; Unassigned; Human |
| | glyceraldehyde-3-phosphate dehydrogenase (GAPDH) mRNA, |
| | complete cds. |
| 12279 | >control:wyeCanine1a:GAPDH3_Mm_at; Unassigned; Mouse |
| | glyceraldehyde-3-phosphate dehydrogenase mRNA, complete cds. |
| 12280 | >control:wyeCanine1a:GAPDH5_Cf_at; GAPDH5 Cf; Cluster |
| | includes AB038240.1 Canis familiaris GAPDH mRNA for |
| | glyceraldehyde-3-phosphate dehydrogenase, complete cds. |
| 12281 | >control:wyeCanine1a:GAPDH5_Hs_at; Unassigned; Human |
| | glyceraldehyde-3-phosphate dehydrogenase (GAPDH) mRNA, |
| | complete cds. |
| 12282 | >control:wyeCanine1a:GAPDH5_Mm_at; Unassigned; Mouse |
| | glyceraldehyde-3-phosphate dehydrogenase mRNA, complete cds. |
| 12283 | >control:wyeCanine1a:GAPDHM_Cf_at; GAPDHM_Cf; Cluster |
| | includes AB038240.1 Canis familiaris GAPDH mRNA for |
| | glyceraldehyde-3-phosphate dehydrogenase, complete cds. |
| 12284 | >control:wyeCanine1a:GAPDHM_Hs_at; Unassigned; Human |
| | glyceraldehyde-3-phosphate dehydrogenase (GAPDH) mRNA, |
| | complete cds. |
| 12285 | >control:wyeCanine1a:GAPDHM_Mm_at; Unassigned; Mouse |
| | glyceraldehyde-3-phosphate dehydrogenase mRNA, complete cds. |
| 12286 | >control:wyeCanine1a:LYSA3_at; Unassigned; Bacillus subtilis |
| | lys gene for diaminopimelate decarboxylase (EC 4.1.1.20). |
| 12287 | >control:wyeCanine1a:LYSA5_at; Unassigned; Bacillus subtilis |
| | lys gene for diaminopimelate decarboxylase (EC 4.1.1.20). |
| 12288 | >control:wyeCanine1a:LYSAM_at; Unassigned; Bacillus subtilis |
| | lys gene for diaminopimelate decarboxylase (EC 4.1.1.20). |
| 12289 | >control:wyeCanine1a:PHE3_at; Unassigned; Bacillus subtillis |
| | phenylalanine biosynthesis associated protein (pheB), and |
| | monofunctional prephenate dehydratase (pheA) genes, complete cds. |
| 12290 | >control:wyeCanine1a:PHE5_at; Unassigned; Bacillus subtillis |
| | phenylalanine biosynthesis associated protein (pheB), and |
| | monofunctional prephenate dehydratase (pheA) genes, complete cds. |
| 12291 | >control:wyeCanine1a:PHEM_at; Unassigned; Bacillus subtillis |
| | phenylalanine biosynthesis associated protein (pheB), and |
| | monofunctional prephenate dehydratase (pheA) genes, complete cds. |
| 12292 | >control:wyeCanine1a:PYRCRB3_Hs_at; Unassigned; Human |
| | pyruvate carboxylase (PC) mRNA, complete cds. |
| 12293 | >control:wyeCanine1a:PYRCRB3_Mm_at; Unassigned; Mus |
| | musculus pyruvate carboxylase mRNA, complete cds. |
| 12294 | >control:wyeCanine1a:PYRCRB5_Hs_at; Unassigned; Human |
| | pyruvate carboxylase (PC) mRNA, complete cds. |
| 12295 | >control:wyeCanine1a:PYRCRB5_Mm_at; Unassigned; Mus |
| | musculus pyruvate carboxylase mRNA, complete cds. |
| 12296 | >control:wyeCanine1a:PYRCRBMA_Hs_at; Unassigned; Human |
| | pyruvate carboxylase (PC) mRNA, complete cds. |
| 12297 | >control:wyeCanine1a:PYRCRBMA_Mm_at; Unassigned; Mus |
| | musculus pyruvate carboxylase mRNA, complete cds. |
| 12298 | >control:wyeCanine1a:PYRCRBMB_Hs_at; Unassigned; Human |
| | pyruvate carboxylase (PC) mRNA, complete cds. |
| 12299 | >control:wyeCanine1a:PYRCRBMB_Mm_at; Unassigned; Mus |
| | musculus pyruvate carboxylase mRNA, complete cds. |
| 12300 | >control:wyeCanine1a:THR3_at; Unassigned; B. subtilis thrB and |
| | thrC genes for homoserine kinase and threonine synthase (EC |
| | 2.7.1.39 and EC 4.2.99.2, respectively). |
| 12301 | >control:wyeCanine1a:THR5_at; Unassigned; B. subtilis thrB and |
| | thrC genes for homoserine kinase and threonine synthase (EC |
| | 2.7.1.39 and EC 4.2.99.2, respectively). |
| 12302 | >control:wyeCanine1a:THRM_at; Unassigned; B. subtilis thrB |
| | and thrC genes for homoserine kinase and threonine synthase (EC |
| | 2.7.1.39 and EC 4.2.99.2, respectively). |
| 12303 | >control:wyeCanine1a:TRANSFR3_Hs_at; Unassigned; Human |
| | transferrin receptor mRNA, complete cds. |
| 12304 | >control:wyeCanine1a:TRANSFR3_Mm_at; Unassigned; |
| | M. musculus mRNA for transferrin receptor. |
| 12305 | >control:wyeCanine1a:TRANSFR5_Hs_at; Unassigned; Human |
| | transferrin receptor mRNA, complete cds. |
| 12306 | >control:wyeCanine1a:TRANSFR5_Mm_at; Unassigned; |
| | M. musculus mRNA for transferrin receptor. |
| 12307 | >control:wyeCanine1a:TRANSFRM_Hs_at; Unassigned; Human |
| | transferrin receptor mRNA, complete cds. |
| 12308 | >control:wyeCanine1a:TRANSFRM_Mm_at; Unassigned; |
| | M. musculus mRNA for transferrin receptor. |
| 12309 | >control:wyeCanine1a:TRP3_at; Unassigned; B. subtilis |
| | tryptophan (trp) operon, complete cds. |
| 12310 | >control:wyeCanine1a:TRP5_at; Unassigned; B. subtilis |
| | tryptophan (trp) operon, complete cds. |
| 12311 | >control:wyeCanine1a:TRPM_at; Unassigned; B. subtilis |
| | tryptophan (trp) operon, complete cds. |

In a preferred embodiment, a nucleic acid array of the present invention comprises a perfect mismatch probe for each polynucleotide probe on the nucleic acid array. A perfect mismatch probe has the same sequence as the original probe except for a homomeric substitution (A to T, T to A, G to C, and C to G) at or near the center of the perfect mismatch probe. For instance, if the original probe has 2n nucleotide residues, the homomeric substitution in the perfect mismatch probe is either at the n or n+1 position, but not at both positions. If the original probe has 2n+1 nucleotide residues, the homomeric substitution in the perfect mismatch probe is at the n+1 position. The center location of the mismatched residue is more likely to destabilize the duplex formed with the target sequence under the hybridization conditions. Each probe and its perfect mismatch probe can be stably attached to different discrete regions on the nucleic acid array.

D. Applications

The nucleic acid arrays of the present invention can be used to detect or monitor gene expression in a vertebrate of interest. The vertebrate can be any animal model of osteoarthritis, such as a canine, a rodent, a rabbit, or a primate. Preferably, the vertebrate is a dog, a rabbit, a rat, a mouse, a hamster, or a guinea pig. The vertebrate can be either non-osteoarthritic, or affected by osteoarthritis. In one specific example, the vertebrate includes both non-osteoarthritic and osteoarthritic cartilage tissues. Other vertebrates can also be analyzed using the nucleic acid arrays of the present invention. In addition, the nucleic acid arrays of the present invention can be used to screen for potential drug candidates or evaluate new therapies for the treatment of osteoarthritis.

Protocols for conducing nucleic acid array hybridization are well known in the art. Exemplary protocols include those provided by Affymetrix in connection with the use of its GeneChip arrays. Samples amenable to nucleic acid array hybridization can be prepared from any tissue of the vertebrate of interest, such as cartilage, heart, liver, kidney, brain, lung, blood, urine, body fluid, or bone marrow. These tissues can be either osteoarthritis-affected or osteoarthritis-free. In one embodiment, the tissue being analyzed is prepared from the same species from which the polynucleotide probes on the nucleic acid array are derived. In another embodiment, the tissue being analyzed is a cartilage tissue prepared from a dog or another animal model of osteoarthritis. As used herein, “tissue” also includes cell preparations or cell cultures. Thus, a cartilage cell preparation or culture is considered a cartilage tissue for the present invention.

The sample for hybridization to a nucleic acid array can be either RNA (e.g., mRNA or cRNA) or DNA (e.g., cDNA). Various methods are available for isolating RNA from tissues. These methods include, but are not limited to, RNeasy kits (provided by QIAGEN), MasterPure kits (provided by Epicentre Technologies), and TRIZOL (provided by Gibco BRL). The RNA isolation protocols provided by Affymetrix can also be used.

The isolated RNA preferably is amplified and/or labeled before being hybridized to a nucleic acid array of the present invention. Suitable RNA amplification methods include, but are not limited to, reverse transcriptase PCR, isothermal amplification, ligase chain reaction, and Qbeta replicase method. The amplification products can be either cDNA or cRNA. In one embodiment, the isolated mRNA is reverse transcribed to cDNA using a reverse transcriptase and a primer consisting of oligo d(T) and a sequence encoding the phage T7 promoter. The cDNA is single stranded. The second strand of the cDNA can be synthesized using a DNA polymerase, combined with an RNase to break up the DNA/RNA hybrid. After synthesis of the double stranded cDNA, T7 RNA polymerase is added to transcribe cRNA from the second strand of the doubled stranded cDNA. The isolated RNA can also be hybridized to a nucleic acid array of the present invention without amplification.

cDNA, cRNA, or other nucleic acid samples can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. The labeling moieties include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Nucleic acid samples can be fragmented before being labeled with detectable moieties. Exemplary methods for fragmentation include, for example, heat and/or ion-mediated hydrolysis.

Hybridization reactions can be performed in absolute or differential hybridization formats. In the absolute hybridization format, polynucleotides derived from one sample are hybridized to the probes in a nucleic acid array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from nucleic acid arrays can be analyzed using commercially available software, such as those provided by Affymetrix or Agilent Technologies. Controls, such as for scan sensitivity, probe labeling and cDNA or cRNA quantitation, are preferably included in the hybridization experiments. Hybridization signals can be scaled or normalized before being subject to further analysis. For instance, hybridization signals for each individual probe can be normalized to take into account variations in hybridization intensities when more than one array is used under similar test conditions. Hybridization signals can also be normalized using the intensities derived from internal normalization controls contained on each array. In addition, genes with relatively consistent expression levels across the samples can be used to normalize the expression levels of other genes. In one embodiment, probes for certain maintenance genes are included in a nucleic acid array of the present invention. These genes are chosen because they show stable levels of expression across a diverse set of tissues. Hybridization signals can be normalized and/or scaled based on the expression levels of these maintenance genes.

In a preferred embodiment, probes for certain exogenous transcripts are included in a nucleic acid array of the present invention. These transcripts can be chosen such that they show no similarity to eukaryotic transcripts. In one specific example, eleven exogenous transcripts at different known concentrations are spiked in to each sample. The array is first scaled to a trimmed-mean target value of 100. Based on the scaled hybridization signal of these eleven probe sets, a standard curve can be drawn such that all transcripts present in the sample can be converted from a signal value to a more meaningful concentration value. In another specific example, a standard curve correlating the signal value read off of the array and known frequency (molarity) can be generated when the array image is read and the probe set expression values are generated. From this standard curve, each signal value can then be converted to a parts per million or picomolarity value. The exogenous controls spiked into each sample can include, for instance, E. coli BioB-5, E. coli BioB-M, E. coli BioB-3, E. coli BioC-5, E. coli BioC-3, E. coli BioD-3, Bacteriophage P1 Cre-5, Bacteriophage P1 Cre-3, E. coli Dap-5, B. subtilis Dap-M, and B. subtilis Dap-3. These transcripts can be monitored by control probe sets as discussed below.

The nucleic acid arrays of the present invention can be used to detect or diagnose osteoarthritis in a vertebrate of interest. A cartilage tissue can be isolated from the vertebrate. RNA samples prepared from the cartilage tissue can be hybridized to a nucleic acid array of the present invention. The expression profiles of genes that are differentially expressed in osteoarthritic tissues as compared to non-osteoarthritic tissues can be used as markers to determine the presence or absence of osteoarthritis in the vertebrate of interest. For instance, an observation showing that the gene expression profile in the cartilage tissue of the vertebrate of interest is more similar to that in osteoarthritic tissues than that in non-osteoarthritic tissues is often indicative of osteoarthritis in the vertebrate.

The nucleic acid arrays of the present invention can also be used to screen for drug candidates or evaluate therapies for treating osteoarthritis or other diseases. In addition, the nucleic acid arrays of the present invention can be used to assess the toxicity or other effects of a drug candidate on dogs or other animals that can be used as models of osteoarthritis.

In one embodiment, a drug candidate is first administered to an osteoarthritic vertebrate. The osteoarthritic vertebrate preferably is an animal model of osteoarthritis, such as a dog. The drug candidate can be formulated in a pharmaceutical composition compatible with the intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

After administration of the drug candidate, a tissue of interest, such as a cartilage tissue, can be isolated from the osteoarthritic vertebrate. A nucleic acid sample is prepared from the tissue and hybridized to a nucleic acid array of the present invention. Preferably, the polynucleotide probes on the nucleic acid array are derived from the same species as the osteoarthritic vertebrate. Any change in hybridization signals after the treatment, compared to that before the treatment, often indicates that the drug candidate can produce a biological response in the osteoarthritic vertebrate (e.g., modulation of expression levels of genes that are differentially expressed in osteoarthritic tissues relative to non-osteoarthritic tissues). Similar methods can be used to assess the effect of a therapy on treating osteoarthritis in dogs or other animal models of osteoarthritis. A return of the expression levels in osteoarthritic tissues to that in osteoarthritis-free tissues is reflective of the efficacy of a drug candidate or therapy in treating osteoarthritis.

In another embodiment, a cartilage tissue or cell culture that mimics a disease state of osteoarthritis is treated with a drug candidate. Nucleic acid molecules can be prepared from the cartilage tissue or cell culture and then hybridized to a nucleic acid array of the present invention. The expression levels of genes that are differentially expressed in osteoarthritic tissues relative to non-osteoarthritic tissues are monitored. A change in the hybridization signals of these genes after the treatment, compared to that before the treatment, is suggestive of the effectiveness of the drug candidate to modulate the expression levels of these differentially expressed genes.

The present invention also features protein arrays for detecting gene expression in animal models of osteoarthritis or other inflammatory diseases. Each protein array of the present invention includes probes which can specifically bind to protein products of genes that are differentially expressed in osteoarthritic cartilage tissues relative to non-osteoarthritic cartilage tissues. Examples of these differentially expressed genes include, but are not limited to, those that encode the tiling sequences selected from Table C and having pattern values of 010, 011, 100 or 101.

In one embodiment, the probes on a protein array of the present invention are antibodies. Many of these antibodies can bind to the corresponding target proteins with an affinity constant of at least 10⁴ M^{-1,} 10⁵ M^{-1,} 10⁶ M⁻¹, 10⁷ M⁻¹, or stronger. Suitable antibodies for the present invention include, but are not limited to, polyclonal antibodies, monoclonal antibodies, chimeric antibodies, single chain antibodies, synthetic antibodies, Fab fragments, or fragments produced by a Fab expression library. Other peptides, scaffolds, antibody mimics, high-affinity binders, or protein-binding ligands can also be used to construct the protein arrays of the present invention.

Numerous methods are available for immobilizing antibodies or other probes on a protein array of the present invention. Examples of these methods include, but are not limited to, diffusion (e.g., agarose or polyacrylamide gel), surface absorption (e.g., nitrocellulose or PVDF), covalent binding (e.g., silanes or aldehyde), or non-covalent affinity binding (e.g., biotin-streptavidin). Examples of protein array fabrication methods include, but are not limited to, ink-jetting, robotic contact printing, photolithography, or piezoelectric spotting. The method described in MacBeath and Schreiber, SCIENCE, 289: 1760-1763 (2000), which is incorporated herein by reference, can also be used. Suitable substrate supports for a protein array of the present invention include, but are not limited to, glass, membranes, mass spectrometer plates, microtiter wells, silica, or beads.

The protein-coding sequence of a gene can be determined by a variety of methods. For instance, the protein-coding sequences can be extracted from the corresponding tiling or parent sequences by using an open reading frame (ORF) prediction program. Examples of ORF prediction programs include, but are not limited to, GeneMark (provided by the European Bioinformatics Institute), Glimmer (provided by TIGR), and ORF Finder (provided by NCBI). Many protein sequences can also be obtained from Entrez or other sequence databases by BLAST searching the corresponding tiling or parent sequences against these databases. The protein-coding sequences thus obtained can be used to prepare antibodies or other protein-binding agents.

In addition, the present invention contemplates a collection of polynucleotides. Each polynucleotide is capable of hybridizing under stringent or nucleic acid array hybridization conditions to a parent sequence selected from SEQ ID NOs: 1-12,167, or the complement thereof. In one embodiment, the polynucleotide collection comprises (1) at least one polynucleotide capable of hybridizing under stringent and/or nucleic acid array hybridization conditions to a tiling sequence selected from Table C and having a pattern value of 010, and (2) at least one additional polynucleotide capable of hybridizing under stringent and/or nucleic acid array hybridization conditions to a tiling sequence selected from Table C and having a pattern value of 100. In another embodiment, the polynucleotide collection includes at least one tiling sequence selected from Table C. For instance, the polynucleotide collection can include at least 5, 10, 50, 100, 500, 1,000, 5,000, 10,000 or more tiling sequences selected from Table C. In yet another embodiment, the polynucleotide collection contains at least 1, 5, 10, 50, 100, 500, 1,000, 5,000, or 10,000 sequence selected from SEQ ID NOs: 1-12,167.

It should be understood that the above-described embodiments and the following examples are given by way of illustration, not limitation. Various changes and modifications within the scope of the present invention will become apparent to those skilled in the art from the present description.

### E. EXAMPLES

### Example 1

### Preparation of Canis familiars Cartilage cDNA Libraries

The cartilage tissue is harvested from non-osteoarthritic or osteoarthritis-affected dogs, and then placed in Dulbecco's Modified Eagle Medium (DMEM, Gibco/BRL) supplemented with antibiotics (penicillin, streptomycin, and gentamicin). The cartilage is removed aseptically from the underlying bone, rinsed in DMEM and diced into small pieces, and then placed in 100 mm petri dishes containing 20 ml of Neuman and Tytell's serum free medium (GIBCO/BRL). Using the protocol of G. Cathala et al., DNA 2:329-335 (1983), the cartilage from each dog is digested with 4 mg/ml pronase (Sigma, St Louis) for 1.5 hours, and then digested with 3 mg/ml bacterial collagenase (Sigma) for 1.5 hours. The digested material is filtered through a cell strainer, and the cells are pelleted by centrifugation. The cell pellet is washed once with phosphate buffered saline and then dissolved in 5 ml of buffer consisting of 5M guanidine isothiocyanate, 10 mM EDTA, 50 mM Tris (pH 7.5) and 8% β-mercaptoethanol. A five-fold volume of 4M LiCl is added to the buffer, and the mixture is stored in the refrigerator overnight. After centrifugation, the precipitate is washed once with 3M LiCl and recentrifuged. The second precipitate is dissolved in a solution consisting of 0.1% sodium dodecyl sulfate, 1 mM EDTA and 10 mM Tris (pH 7.5). The suspension is frozen at −70° C. and then vortexed during thawing.

Total RNA is extracted twice with phenol chloroform, once with chloroform, and then precipitated with ethanol. Following centrifugation, the RNA pellet is redissolved in DEPC treated, distilled deionized water (DEPC-ddHOH) and run over a CsCl gradient. The RNA is extracted with acid phenol (at pH 4.0, catalog #972Z, Ambion, Austin Tex.), precipitated with ethanol and resuspended in DEPC-ddHOH. The RNA is treated with RNase-free DNase (Epicentre Technologies, Madison Wis.) for 15 minutes, extracted with chloroform, precipitated and washed with ethanol, and dissolved in DEPC-ddHOH. Since the RNA yield may vary with each sample, approximately 40% of total RNA from each sample is contributed to the pooled sample. The pooled RNA is used to construct a cartilage cDNA library.

### Example 2.

### Analysis of Cartilage cDNA Libraries

To assess the quality of the Canis familiars cartilage cDNA libraries, 379 3′ sequence reads were produced from an osteoarthritis-affected cartilage cDNA library, and 432 3′ sequence reads were generated from an non-osteoarthritic cartilage cDNA library. Each of the 3′ sequence reads was compared by BLASTN to both the human genome as well as the human sequences present in GenBank. Table H shows the BLASTN results for the 3′ sequence reads from the osteoarthritis-free library (“Free”) as well as from the osteoarthritis-affected library (“Affected”). Those 3′ sequence reads that were mapped to a human cDNA with an expect (“e-”) value no less than 10⁻¹⁴ were considered to be homologous to the human transcript.

TABLE H
Analysis of Cartilage cDNA Libraries

| | | | | A-F |
|---|---|---|---|---|
| Gene Symbol | Gene Name | Free | Affected | Change |
| GP | matrix Gla protein | 7 | 0 | −7 |
| RPL10 | ribosomal protein L10 | 8 | 2 | −6 |
| RPS3A | ribosomal protein S3A | 6 | 1 | −5 |
| CHAD | chondroadherin | 4 | 1 | −3 |
| CLU | clusterin (complement lysis inhibitor, SP- | 6 | 3 | −3 |
| | 40, 40, sulfated glycoprotein 2, | | | |
| | testosterone-repressed prostate message | | | |
| | 2, apolipoprotein J) | | | |
| CST3 | cystatin C (amyloid angiopathy and | 3 | 0 | −3 |
| | cerebral hemorrhage) | | | |
| RPL10A | ribosomal protein L10a | 3 | 0 | −3 |
| RPL8 | ribosomal protein L8 | 3 | 0 | −3 |
| RPS8 | ribosomal protein S8 | 3 | 0 | −3 |
| UNK_AK023362 | UNKNOWN | 3 | 0 | −3 |
| UNK_K03432 | UNKNOWN | 5 | 2 | −3 |
| BTF3 | basic transcription factor 3 | 2 | 0 | −2 |
| K-ALPHA-1 | tubulin, alpha, ubiquitous | 2 | 0 | −2 |
| PRO1073 | PRO1073 protein | 2 | 0 | −2 |
| RPL4 | ribosomal protein L4 | 2 | 0 | −2 |
| RPLP0 | ribosomal protein, large, P0 | 3 | 1 | −2 |
| RPS11 | ribosomal protein S11 | 2 | 0 | −2 |
| 13CDNA73 | hypothetical protein CG003 | 1 | 0 | −1 |
| ACTG1 | actin, gamma 1 | 2 | 1 | −1 |
| ADRM1 | adhesion regulating molecule 1 | 1 | 0 | −1 |
| AMOTL2 | angiomotin like 2 | 1 | 0 | −1 |
| ANXA5 | annexin A5 | 1 | 0 | −1 |
| APRT | adenine phosphoribosyltransferase | 1 | 0 | −1 |
| ARF3 | ADP-ribosylation factor 3 | 1 | 0 | −1 |
| ARPC1B | actin related protein 2/3 complex, subunit | 1 | 0 | −1 |
| | 1B, 41 kDa | | | |
| ATP6V1D | ATPase, H+ transporting, lysosomal | 1 | 0 | −1 |
| | 34 kDa, V1 subunit D | | | |
| BTBD1 | BTB (POZ) domain containing 1 | 1 | 0 | −1 |
| BTG2 | BTG family, member 2 | 1 | 0 | −1 |
| CABC1 | chaperone, ABC1 activity of bc1 | 1 | 0 | −1 |
| | complex like (S. pombe) | | | |
| CCK | cholecystokinin | 1 | 0 | −1 |
| COL10A1 | collagen, type X, alpha 1(Schmid | 1 | 0 | −1 |
| | metaphyseal chondrodysplasia) | | | |
| COX7A2L | cytochrome c oxidase subunit VIIa | 1 | 0 | −1 |
| | polypeptide 2 like | | | |
| CTSL2 | cathepsin L2 | 1 | 0 | −1 |
| D21S2056E | DNA segment on chromosome 21 | 1 | 0 | −1 |
| | (unique) 2056 expressed sequence | | | |
| DDOST | dolichyl-diphosphooligosaccharide- | 1 | 0 | −1 |
| | protein glycosyltransferase | | | |
| DKFZP566H073 | DKFZP566H073 protein | 1 | 0 | −1 |
| DMPK | dystrophia myotonica-protein kinase | 1 | 0 | −1 |
| EIF3S4 | eukaryotic translation initiation factor 3, | 1 | 0 | −1 |
| | subunit 4 delta, 44 kDa | | | |
| EIF4G2 | eukaryotic translation initiation factor 4 | 1 | 0 | −1 |
| | gamma, 2 | | | |
| ERH | enhancer of rudimentary homolog | 1 | 0 | −1 |
| | (Drosophila) | | | |
| F13A1 | coagulation factor XIII, A1 polypeptide | 1 | 0 | −1 |
| FLJ13081 | hypothetical protein FLJ13081 | 1 | 0 | −1 |
| FLJ22729 | hypothetical protein FLJ22729 | 1 | 0 | −1 |
| FOSL2 | FOS-like antigen 2 | 1 | 0 | −1 |
| FTH1 | ferritin, heavy polypeptide 1 | 3 | 2 | −1 |
| GDF10 | growth differentiation factor 10 | 1 | 0 | −1 |
| GSN | gelsolin (amyloidosis, Finnish type) | 1 | 0 | −1 |
| HBS1L | HBS1-like (S. cerevisiae) | 1 | 0 | −1 |
| HSPC163 | HSPC163 protein | 1 | 0 | −1 |
| ID1 | inhibitor of DNA binding 1, dominant | 1 | 0 | −1 |
| | negative helix-loop-helix protein | | | |
| IMPDH2 | IMP (inosine monophosphate) | 1 | 0 | −1 |
| | dehydrogenase 2 | | | |
| KIAA0375 | KIAA0375 gene product | 1 | 0 | −1 |
| KIAA1053 | KIAA1053 protein | 1 | 0 | −1 |
| LAMR1 | laminin receptor 1 (ribosomal protein SA, | 3 | 2 | −1 |
| | 67 kDa) | | | |
| LOC119504 | hypothetical protein LOC119504 | 1 | 0 | −1 |
| MDH1 | malate dehydrogenase 1, NAD (soluble) | 1 | 0 | −1 |
| METAP2 | methionyl aminopeptidase 2 | 1 | 0 | −1 |
| MGC10471 | hypothetical protein MGC10471 | 1 | 0 | −1 |
| MGC13017 | similar to RIKEN cDNA A430101B06 | 1 | 0 | −1 |
| | gene | | | |
| MGC20781 | hypothetical protein MGC20781 | 1 | 0 | −1 |
| MGC3035 | hypothetical protein MGC3035 | 1 | 0 | −1 |
| MPG | N-methylpurine-DNA glycosylase | 1 | 0 | −1 |
| MRPS18B | mitochondrial ribosomal protein S18B | 1 | 0 | −1 |
| MRPS31 | mitochondrial ribosomal protein S31 | 1 | 0 | −1 |
| MYL6 | myosin, light polypeptide 6, alkali, | 1 | 0 | −1 |
| | smooth muscle and non-muscle | | | |
| NACA | nascent-polypeptide-associated complex | 1 | 0 | −1 |
| | alpha polypeptide | | | |
| PAI-RBP1 | PAI-1 mRNA-binding protein | 1 | 0 | −1 |
| POLR1C | polymerase (RNA) I polypeptide C, | 1 | 0 | −1 |
| | 30 kDa | | | |
| PSMB3 | proteasome (prosome, macropain) | 1 | 0 | −1 |
| | subunit, beta type, 3 | | | |
| PYGL | phosphorylase, glycogen; liver (Hers | 1 | 0 | −1 |
| | disease, glycogen storage disease type | | | |
| | VI) | | | |
| RABAC1 | Rab acceptor 1 (prenylated) | 1 | 0 | −1 |
| RNF130 | ring finger protein 130 | 1 | 0 | −1 |
| RNF5 | ring finger protein 5 | 1 | 0 | −1 |
| RPC62 | polymerase (RNA) III (DNA directed) | 1 | 0 | −1 |
| | (62 kD) | | | |
| RPL10L | ribosomal protein L10-like | 1 | 0 | −1 |
| RPL12 | ribosomal protein L12 | 1 | 0 | −1 |
| RPL13A | ribosomal protein L13a | 2 | 1 | −1 |
| RPL17 | ribosomal protein L17 | 1 | 0 | −1 |
| RPL18 | ribosomal protein L18 | 1 | 0 | −1 |
| RPL19 | ribosomal protein L19 | 1 | 0 | −1 |
| RPL21 | ribosomal protein L21 | 1 | 0 | −1 |
| RPL34 | ribosomal protein L34 | 1 | 0 | −1 |
| RPL6 | ribosomal protein L6 | 1 | 0 | −1 |
| RPL9 | ribosomal protein L9 | 2 | 1 | −1 |
| RPS6 | ribosomal protein S6 | 2 | 1 | −1 |
| SAA1 | serum amyloid A1 | 1 | 0 | −1 |
| SCP2 | sterol carrier protein 2 | 1 | 0 | −1 |
| SEC23B | Sec23 homolog B (S. cerevisiae) | 1 | 0 | −1 |
| SERF1A | small EDRK-rich factor 1A (telomeric) | 1 | 0 | −1 |
| SERPINA1 | serine (or cysteine) proteinase inhibitor, | 3 | 2 | −1 |
| | clade A (alpha-1 antiproteinase, | | | |
| | antitrypsin), member 1 | | | |
| SLC25A6 | solute carrier family 25 (mitochondrial | 1 | 0 | −1 |
| | carrier; adenine nucleotide translocator), | | | |
| | member 6 | | | |
| SREBF1 | sterol regulatory element binding | 1 | 0 | −1 |
| | transcription factor 1 | | | |
| SRP72 | signal recognition particle 72 kDa | 1 | 0 | −1 |
| STRAIT11499 | hypothetical protein STRAIT11499 | 1 | 0 | −1 |
| SUI1 | putative translation initiation factor | 1 | 0 | −1 |
| TGFBR3 | transforming growth factor, beta receptor | 1 | 0 | −1 |
| | III (betaglycan, 300 kDa) | | | |
| THBS1 | thrombospondin 1 | 1 | 0 | −1 |
| THY1 | Thy-1 cell surface antigen | 1 | 0 | −1 |
| TIMP1 | tissue inhibitor of metalloproteinase 1 | 1 | 0 | −1 |
| | (erythroid potentiating activity, | | | |
| | collagenase inhibitor) | | | |
| TLN1 | talin 1 | 1 | 0 | −1 |
| TPM1 | tropomyosin 1 (alpha) | 1 | 0 | −1 |
| UBAP2 | ubiquitin associated protein 2 | 1 | 0 | −1 |
| UBB | ubiquitin B | 1 | 0 | −1 |
| UBC | ubiquitin C | 1 | 0 | −1 |
| UNK_AK000896 | UNKNOWN | 1 | 0 | −1 |
| UNK_AK025781 | Homo sapiens, clone IMAGE: 5211207, | 1 | 0 | −1 |
| | mRNA | | | |
| UNK_AK026491 | UNKNOWN | 1 | 0 | −1 |
| UNK_AK054605 | UNKNOWN | 1 | 0 | −1 |
| UNK_AK057071 | UNKNOWN | 4 | 3 | −1 |
| UNK_BC014023 | UNKNOWN | 1 | 0 | −1 |
| UNK_BC017189 | UNKNOWN | 1 | 0 | −1 |
| UNK_U37146 | UNKNOWN | 1 | 0 | −1 |
| UNK_U76194 | UNKNOWN | 1 | 0 | −1 |
| VAPB | VAMP (vesicle-associated membrane | 1 | 0 | −1 |
| | protein)-associated protein B and C | | | |
| VPS28 | vacuolar protein sorting 28 (yeast) | 1 | 0 | −1 |
| WDR1 | WD repeat domain 1 | 1 | 0 | −1 |
| WDR5 | WD repeat domain 5 | 1 | 0 | −1 |
| WIF1 | WNT inhibitory factor 1 | 1 | 0 | −1 |
| WIZ | widely-interspaced zinc finger motifs | 1 | 0 | −1 |
| YWHAE | tyrosine 3-monooxygenase/tryptophan 5- | 1 | 0 | −1 |
| | monooxygenase activation protein, | | | |
| | epsilon polypeptide | | | |
| ZNF236 | zinc finger protein 236 | 1 | 0 | −1 |
| ZNF-U69274 | zinc finger protein | 1 | 0 | −1 |
| DCN | decorin | 1 | 1 | 0 |
| DNASE1L1 | deoxyribonuclease I-like 1 | 1 | 1 | 0 |
| ECRG4 | esophageal cancer related gene 4 protein | 2 | 2 | 0 |
| EEF1A1 | eukaryotic translation elongation factor 1 | 2 | 2 | 0 |
| | alpha 1 | | | |
| EEF1B2 | eukaryotic translation elongation factor 1 | 1 | 1 | 0 |
| | beta 2 | | | |
| FGFR1 | fibroblast growth factor receptor 1 (fms- | 1 | 1 | 0 |
| | related tyrosine kinase 2, Pfeiffer | | | |
| | syndrome) | | | |
| H3F3B | H3 histone, family 3B (H3.3B) | 1 | 1 | 0 |
| ITGB5 | integrin, beta 5 | 1 | 1 | 0 |
| MGC3047 | hypothetical protein MGC3047 | 1 | 1 | 0 |
| PIR | Pirin | 1 | 1 | 0 |
| PSMA2 | proteasome (prosome, macropain) | 1 | 1 | 0 |
| | subunit, alpha type, 2 | | | |
| RPL7 | ribosomal protein L7 | 1 | 1 | 0 |
| RPS2 | ribosomal protein S2 | 1 | 1 | 0 |
| TPT1 | tumor protein, translationally-controlled 1 | 2 | 2 | 0 |
| TSPAN-3 | tetraspan 3 | 1 | 1 | 0 |
| UNK_AJ328465 | UNKNOWN | 1 | 1 | 0 |
| ACTA1 | actin, alpha 1, skeletal muscle | 0 | 1 | 1 |
| AGC1 | aggrecan 1 (chondroitin sulfate | 0 | 1 | 1 |
| | proteoglycan 1, large aggregating | | | |
| | proteoglycan, antigen identified by | | | |
| | monoclonal antibody A0122) | | | |
| AP2M1 | adaptor-related protein complex 2, mu 1 | 0 | 1 | 1 |
| | subunit | | | |
| APOE | apolipoprotein E | 0 | 1 | 1 |
| AQP1 | aquaporin 1 (channel-forming integral | 0 | 1 | 1 |
| | protein, 28 kDa) | | | |
| ARHA | ras homolog gene family, member A | 0 | 1 | 1 |
| ASS | argininosuccinate synthetase | 0 | 1 | 1 |
| BCKDHB | branched chain keto acid dehydrogenase | 0 | 1 | 1 |
| | E1, beta polypeptide (maple syrup urine | | | |
| | disease) | | | |
| BGN | biglycan | 0 | 1 | 1 |
| BOC | brother of CDO | 0 | 1 | 1 |
| C13ORF9 | chromosome 13 open reading frame 9 | 0 | 1 | 1 |
| C1ORF8 | chromosome 1 open reading frame 8 | 0 | 1 | 1 |
| C1QTNF7 | C1q and tumor necrosis factor related | 0 | 1 | 1 |
| | protein 7 | | | |
| C20ORF67 | chromosome 20 open reading frame 67 | 0 | 1 | 1 |
| C5ORF3 | chromosome 5 open reading frame 3 | 0 | 1 | 1 |
| C6ORF37 | chromosome 6 open reading frame 37 | 0 | 1 | 1 |
| CALM2 | calmodulin 2 (phosphorylase kinase, | 0 | 1 | 1 |
| | delta) | | | |
| CAPN2 | calpain 2, (m/II) large subunit | 0 | 1 | 1 |
| CD164 | CD164 antigen, sialomucin | 0 | 1 | 1 |
| CD81 | CD81 antigen (target of antiproliferative | 0 | 1 | 1 |
| | antibody 1) | | | |
| CGI-135 | CGI-135 protein | 0 | 1 | 1 |
| CGI-148 | CGI-148 protein | 0 | 1 | 1 |
| CGI-99 | CGI-99 protein | 0 | 1 | 1 |
| CHST1 | carbohydrate (keratan sulfate Gal-6) | 0 | 1 | 1 |
| | sulfotransferase 1 | | | |
| CKM | creatine kinase, muscle | 0 | 1 | 1 |
| COL11A2 | collagen, type XI, alpha 2 | 0 | 1 | 1 |
| COL12A1 | collagen, type XII, alpha 1 | 0 | 1 | 1 |
| COL1A1 | collagen, type I, alpha 1 | 0 | 1 | 1 |
| COL3A1 | collagen, type III, alpha 1 (Ehlers-Danlos | 0 | 1 | 1 |
| | syndrome type IV, autosomal dominant) | | | |
| COPE | coatomer protein complex, subunit | 0 | 1 | 1 |
| | epsilon | | | |
| CRABP2 | cellular retinoic acid binding protein 2 | 0 | 1 | 1 |
| CRIM1 | cysteine-rich motor neuron 1 | 0 | 1 | 1 |
| CRSP6 | cofactor required for Sp1 transcriptional | 0 | 1 | 1 |
| | activation, subunit 6, 77 kDa | | | |
| DDB2 | damage-specific DNA binding protein 2, | 0 | 1 | 1 |
| | 48 kDa | | | |
| DKFZP564O092 | DKFZP564O092 protein | 0 | 1 | 1 |
| DKFZP566E144 | small fragment nuclease | 0 | 1 | 1 |
| DKKL1-PENDING | soggy-1 gene | 0 | 1 | 1 |
| DLC1 | deleted in liver cancer 1 | 0 | 1 | 1 |
| DP1 | likely ortholog of mouse deleted in | 0 | 1 | 1 |
| | polyposis 1 | | | |
| E1B-AP5 | E1B-55 kDa-associated protein 5 | 0 | 1 | 1 |
| EEF1D | eukaryotic translation elongation factor 1 | 2 | 3 | 1 |
| | delta (guanine nucleotide exchange | | | |
| | protein) | | | |
| EEF1G | eukaryotic translation elongation factor 1 | 0 | 1 | 1 |
| | gamma | | | |
| EIF3K | eukaryotic translation initiation factor 3 | 0 | 1 | 1 |
| | subunit k | | | |
| EIF4A2 | eukaryotic translation initiation factor | 0 | 1 | 1 |
| | 4A, isoform 2 | | | |
| ELF1 | E74-like factor 1 (ets domain | 0 | 1 | 1 |
| | transcription factor) | | | |
| EMP3 | epithelial membrane protein 3 | 0 | 1 | 1 |
| ENO1 | enolase 1, (alpha) | 0 | 1 | 1 |
| ETFB | electron-transfer-flavoprotein, beta | 0 | 1 | 1 |
| | polypeptide | | | |
| FBXW1B | F-box and WD-40 domain protein 1B | 0 | 1 | 1 |
| FKBP1A | FK506 binding protein 1A, 12 kDa | 0 | 1 | 1 |
| FLJ11171 | hypothetical protein FLJ11171 | 0 | 1 | 1 |
| FLJ23751 | hypothetical protein FLJ23751 | 0 | 1 | 1 |
| FOLR2 | folate receptor 2 (fetal) | 0 | 1 | 1 |
| GABARAP | GABA(A) receptor-associated protein | 0 | 1 | 1 |
| GEMIN6 | gem (nuclear organelle) associated | 0 | 1 | 1 |
| | protein 6 | | | |
| GLG1 | golgi apparatus protein 1 | 0 | 1 | 1 |
| GNA11 | guanine nucleotide binding protein (G | 0 | 1 | 1 |
| | protein), alpha 11 (Gq class) | | | |
| GNAI2 | guanine nucleotide binding protein (G | 0 | 1 | 1 |
| | protein), alpha inhibiting activity | | | |
| | polypeptide 2 | | | |
| GPX3 | glutathione peroxidase 3 (plasma) | 0 | 1 | 1 |
| GSTM3 | glutathione S-transferase M3 (brain) | 0 | 1 | 1 |
| H17 | hypothetical protein H17 | 0 | 1 | 1 |
| HADH2 | hydroxyacyl-Coenzyme A | 0 | 1 | 1 |
| | dehydrogenase, type II | | | |
| HDAC6 | histone deacetylase 6 | 0 | 1 | 1 |
| HHGP | HHGP protein | 0 | 1 | 1 |
| HNOEL-ISO | HNOEL-iso protein | 0 | 1 | 1 |
| HNRPA1 | heterogeneous nuclear ribonucleoprotein | 0 | 1 | 1 |
| | A1 | | | |
| HSPA1A | heat shock 70 kDa protein 1A | 0 | 1 | 1 |
| HSPC039 | HSPC039 protein | 0 | 1 | 1 |
| IDUA | iduronidase, alpha-L- | 0 | 1 | 1 |
| ING4 | inhibitor of growth family, member 4 | 0 | 1 | 1 |
| LASP1 | LIM and SH3 protein 1 | 0 | 1 | 1 |
| LDHA | lactate dehydrogenase A | 0 | 1 | 1 |
| LENG1 | leukocyte receptor cluster (LRC) member 1 | 0 | 1 | 1 |
| LOC283824 | hypothetical protein LOC283824 | 0 | 1 | 1 |
| LOC286257 | hypothetical protein LOC286257 | 0 | 1 | 1 |
| LOC56270 | hypothetical protein 628 | 0 | 1 | 1 |
| LRP10 | low density lipoprotein receptor-related | 0 | 1 | 1 |
| | protein 10 | | | |
| MAGED1 | melanoma antigen, family D, 1 | 0 | 1 | 1 |
| MGC10540 | hypothetical protein MGC10540 | 0 | 1 | 1 |
| MGC2306 | hypothetical protein MGC2306 | 0 | 1 | 1 |
| MLF2 | myeloid leukemia factor 2 | 0 | 1 | 1 |
| MORF4L2 | mortality factor 4 like 2 | 0 | 1 | 1 |
| MTX1 | metaxin 1 | 0 | 1 | 1 |
| MVP | major vault protein | 0 | 1 | 1 |
| MYH10 | myosin, heavy polypeptide 10, non- | 0 | 1 | 1 |
| | muscle | | | |
| MYL2 | myosin, light polypeptide 2, regulatory, | 0 | 1 | 1 |
| | cardiac, slow | | | |
| MYST3 | MYST histone acetyltransferase | 0 | 1 | 1 |
| | (monocytic leukemia) 3 | | | |
| NDP52 | nuclear domain 10 protein | 0 | 1 | 1 |
| NDUFV1 | NADH dehydrogenase (ubiquinone) | 0 | 1 | 1 |
| | flavoprotein 1, 51 kDa | | | |
| NESHBP | DKFZP586L2024 protein | 0 | 1 | 1 |
| NPDC1 | neural proliferation, differentiation and | 0 | 1 | 1 |
| | control, 1 | | | |
| NR1D1 | nuclear receptor subfamily 1, group D, | 0 | 1 | 1 |
| | member 1 | | | |
| NR4A2 | nuclear receptor subfamily 4, group A, | 0 | 1 | 1 |
| | member 2 | | | |
| NSEP1 | nuclease sensitive element binding | 0 | 1 | 1 |
| | protein 1 | | | |
| OAZ2 | ornithine decarboxylase antizyme 2 | 0 | 1 | 1 |
| ORF1-FL49 | putative nuclear protein ORF1-FL49 | 0 | 1 | 1 |
| P4HA1 | procollagen-proline, 2-oxoglutarate 4- | 0 | 1 | 1 |
| | dioxygenase (proline 4-hydroxylase), | | | |
| | alpha polypeptide I | | | |
| P5 | protein disulfide isomerase-related | 0 | 1 | 1 |
| | protein | | | |
| PABPC4 | poly(A) binding protein, cytoplasmic 4 | 0 | 1 | 1 |
| | (inducible form) | | | |
| PCCB | propionyl Coenzyme A carboxylase, beta | 0 | 1 | 1 |
| | polypeptide | | | |
| PDK1 | pyruvate dehydrogenase kinase, | 0 | 1 | 1 |
| | isoenzyme 1 | | | |
| PDK4 | pyruvate dehydrogenase kinase, | 0 | 1 | 1 |
| | isoenzyme 4 | | | |
| PMP22 | peripheral myelin protein 22 | 0 | 1 | 1 |
| PMPCB | peptidase (mitochondrial processing) beta | 0 | 1 | 1 |
| POLR2E | polymerase (RNA) II (DNA directed) | 0 | 1 | 1 |
| | polypeptide E, 25 kDa | | | |
| PPP1R14B | protein phosphatase 1, regulatory | 0 | 1 | 1 |
| | (inhibitor) subunit 14B | | | |
| PRDX1 | peroxiredoxin 1 | 0 | 1 | 1 |
| PSMD8 | proteasome (prosome, macropain) 26S | 0 | 1 | 1 |
| | subunit, non-ATPase, 8 | | | |
| PTD008 | PTD008 protein | 0 | 1 | 1 |
| RAI16 | retinoic acid induced 16 | 0 | 1 | 1 |
| RBMX | RNA binding motif protein, X | 0 | 1 | 1 |
| | chromosome | | | |
| RELA | v-rel reticuloendotheliosis viral oncogene | 0 | 1 | 1 |
| | homolog A, nuclear factor of kappa light | | | |
| | polypeptide gene enhancer in B-cells 3, | | | |
| | p65 (avian) | | | |
| RGC32 | RGC32 protein | 0 | 1 | 1 |
| RPL7A | ribosomal protein L7a | 1 | 2 | 1 |
| SCAND1 | SCAN domain containing 1 | 0 | 1 | 1 |
| SEC24B | SEC24 related gene family, member B | 0 | 1 | 1 |
| | (S. cerevisiae) | | | |
| SIL1 | endoplasmic reticulum chaperone SIL1, | 0 | 1 | 1 |
| | homolog of yeast | | | |
| SLC25A4 | solute carrier family 25 (mitochondrial | 0 | 1 | 1 |
| | carrier; adenine nucleotide translocator), | | | |
| | member 4 | | | |
| SMOC2 | SPARC related modular calcium binding 2 | 0 | 1 | 1 |
| SMYD5 | SMYD family member 5 | 0 | 1 | 1 |
| SPOP | speckle-type POZ protein | 0 | 1 | 1 |
| TERF2IP | telomeric repeat binding factor 2, | 0 | 1 | 1 |
| | interacting protein | | | |
| TINP1 | TGF beta-inducible nuclear protein 1 | 0 | 1 | 1 |
| TLP19 | thioredoxin-like protein p19 | 0 | 1 | 1 |
| TRIM28 | tripartite motif-containing 28 | 0 | 1 | 1 |
| TRPV4 | transient receptor potential cation | 0 | 1 | 1 |
| | channel, subfamily V, member 4 | | | |
| UNK_AF031379 | UNKNOWN | 0 | 1 | 1 |
| UNK_BC005228 | UNKNOWN | 0 | 1 | 1 |
| UNK_BC007204 | Homo sapiens, clone IMAGE: 4815142, | 0 | 1 | 1 |
| | mRNA | | | |
| UNK_BC009736 | UNKNOWN | 0 | 1 | 1 |
| UNK_D29011 | UNKNOWN | 0 | 1 | 1 |
| UNK_M63394 | UNKNOWN | 0 | 1 | 1 |
| UNK_X07868 | UNKNOWN | 0 | 1 | 1 |
| UXT | ubiquitously-expressed transcript | 0 | 1 | 1 |
| VPS26 | vacuolar protein sorting 26 (yeast) | 0 | 1 | 1 |
| WBP2 | WW domain binding protein 2 | 0 | 1 | 1 |
| FN1 | fibronectin 1 | 1 | 3 | 2 |
| GAPD | glyceraldehyde-3-phosphate | 2 | 4 | 2 |
| | dehydrogenase | | | |
| HSPCB | heat shock 90 kDa protein 1, beta | 0 | 2 | 2 |
| LUM | lumican | 0 | 2 | 2 |
| UNK_AF035455 | UNKNOWN | 0 | 2 | 2 |
| UNK_M14219 | UNKNOWN | 2 | 4 | 2 |
| UNK_X02670 | UNKNOWN | 0 | 2 | 2 |
| VIM | vimentin | 2 | 4 | 2 |
| FMOD | fibromodulin | 0 | 3 | 3 |
| RPS4X | ribosomal protein S4, X-linked | 3 | 6 | 3 |
| SPARC | secreted protein, acidic, cysteine-rich | 3 | 6 | 3 |
| | (osteonectin) | | | |
| UNK_AF227907 | UNKNOWN | 0 | 3 | 3 |
| CLECSF1 | C-type (calcium dependent, | 1 | 5 | 4 |
| | carbohydrate-recognition domain) lectin, | | | |
| | superfamily member 1 (cartilage-derived) | | | |
| RPS5 | ribosomal protein S5 | 1 | 5 | 4 |
| COMP | cartilage oligomeric matrix protein | 3 | 10 | 7 |
| | (pseudoachondroplasia, epiphyseal | | | |
| | dysplasia 1, multiple) | | | |
| COL2A1 | collagen, type II, alpha 1 (primary | 0 | 9 | 9 |
| | osteoarthritis, spondyloepiphyseal | | | |
| | dysplasia, congenital) | | | |

Of the 379 non-osteoarthritic 3′ sequence reads, about 58.6% were found to be homologous with a human transcript. Of the 432 osteoarthritis-affected 3′ sequence reads, 58.1% were found to be homologous with a human transcript.

Given the homology mappings above, a simple in silico transcription profiling experiment was conducted. As shown above, each canine 3′ read was mapped to a human transcript. These human transcripts are collected into two distinct gene indices at the NCBI, Unigene and LocusLink. Using these gene indices, all of the mapped 3′ reads were associated to their corresponding genes (i.e., the genes under “Gene Symbol” and “Gene Name”). By doing a simple count of each gene in the osteoarthritis-free and osteoarthritis-affected libraries, some indications as to the transcription profile in the non-osteoarthritic and osteoarthritis-affected tissues can be obtained. For example, the osteoarthritis-free library contains seven copies of the matrix Gla protein, whereas the affected library contains zero copies. Conversely, the affected library contains nine copies of the collagen, type II, alpha 1 protein, whereas the normal library contains zero copies. The ratio of the frequency of occurrence of a 3′ sequence read in the osteoarthritis-affected library over that in the osteoarthritis-free library is shown under “A-F Change.”

This analysis has been performed on a small set of data. Similar analysis can be applied to a larger set of 3′ sequence reads, as appreciated by those skilled in the art.

### Example 3.

### Nucleic Acid Array

The tiling sequences depicted in Table C were submitted to Affymetrix for custom array design. Affymetrix selected probes for each tiling sequence using its probe-picking algorithm. Non-ambiguous probes with 25 bases in length were selected. Sixteen probe-pairs were requested for each tiling sequence with a minimum number of acceptable probe-pairs set to twelve. The final array was directed to 11,986 Canis familiars transcripts and contained 197,796 perfect match probes and 197,796 mismatch probes, including 137 exogenous control probe sets. These probes are shown in Table I.

The probes in Table I are perfect match probes and correspond to SEQ ID NOs: 12,312-210,107, respectively. Each probe in Table I has a qualifier which is identical to the qualifier of the corresponding tiling sequence from which the probe is derived. The strandedness of each probe (“Direction”) is also demonstrated.

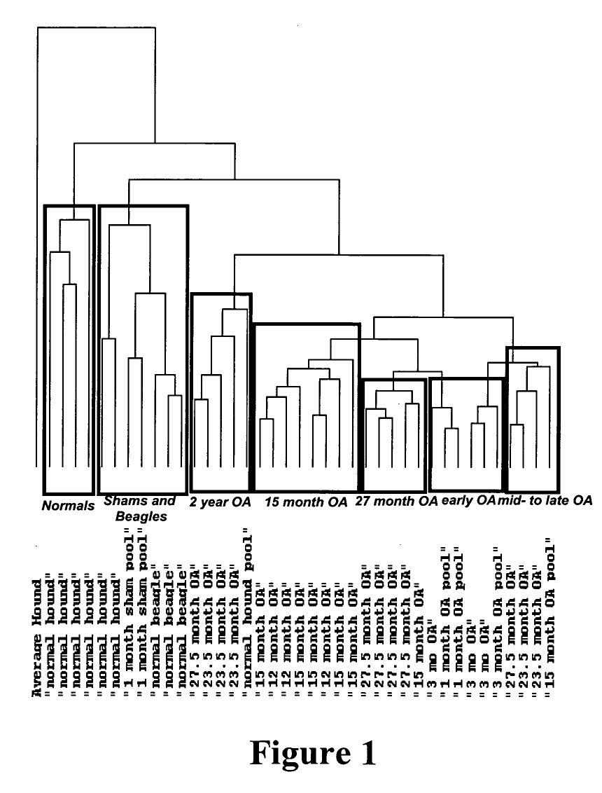

FIG. 1 represents an Eisen cluster of transcriptional profiling data generated using the above-described custom array. Data was scale frequency normalized. Only those qualifiers with at least 1 present call in any sample were used for the cluster analysis. Data were log transformed, and hierarchical clustering was done using the complete linkage clustering finction on the arrays. Levels of all expressed genes strongly segregate canine osteoartritis (OA) samples by temporal stage of disease.

### Example 4.

### Nucleic Acid Array Hybridization

10 μg of biotin-labeled sample DNA/RNA is diluted in 1× MES buffer with 100 μg/ml herring sperm DNA and 50 μg/ml acetylated BSA. To normalize arrays to each other and to estimate the sensitivity of the nucleic acid arrays, in vitro synthesized transcripts of control genes are included in each hybridization reaction. The abundance of these transcripts can range from 1:300,000 (3 ppm) to 1:1000 (1000 ppm) stated in terms of the number of control transcripts per total transcripts. As determined by the signal response from these control transcripts, the sensitivity of detection of the arrays can range, for example, between about 1:300,000 and 1:100,000 copies/million. Labeled DNA/RNA are denatured at 99° C. for 5 minutes and then 45° C. for 5 minutes and hybridized to the nucleic array of Example 3. The array is hybridized for 16 hours at 45° C. The hybridization buffer includes 100 mM. MES, 1 M [Na+], 20 mM EDTA, and 0.01% Tween 20. After hybridization, the cartridge(s) is washed extensively with wash buffer (6×SSPET), for instance, three 10-minute washes at room temperature. The washed cartridge(s) is then stained with phycoerythrin coupled to streptavidin.

12× MES stock contains 1.22 M MES and 0.89 M [Na+]. For 1000 ml, the stock can be prepared by mixing 70.4 g MES free acid monohydrate, 193.3 g MES sodium salt and 800 ml of molecular biology grade water, and adjusting volume to 1000 ml. The pH should be between 6.5 and 6.7. 2× hybridization buffer can be prepared by mixing 8.3 ml of 12× MES stock, 17.7 ml of 5 M NaCl, 4.0 ml of 0.5 M EDTA, 0.1 ml of 10% Tween 20 and 19.9 ml of water. 6× SSPET contains 0.9 M NaCl, 60 mM NaH₂PO₄, 6 mM EDTA, pH 7.4, and 0.005% Triton X-100. In some cases, the wash buffer can be replaced with a more stringent wash buffer. 1000 ml stringent wash buffer can be prepared by mixing 83.3 ml of 12× MES stock, 5.2 ml of 5 M NaCl, 1.0 ml of 10% Tween 20 and 910.5 ml of water.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. A nucleic acid array comprising at least one polynucleotide probe capable of hybridizing under stringent or nucleic acid array hybridization conditions to a gene which is differentially expressed in osteoarthritic cartilage cells relative to non-osteoarthritic cartilage cells, and wherein said osteoarthritic and non-osteoarthritic cartilage cells are derived from the same species.

2. The nucleic acid array according to claim 1, comprising at least 10 polynucleotide probe sets, wherein each said probe set is capable of hybridizing under, stringent or nucleic acid array hybridization conditions to a different respective gene which is differentially expressed in said osteoartlritic cartilage cells relative to said non-osteoarthritic cartilage cells.

3. The nucleic acid array according to claim 1, comprising at least 100 polynucleotide probe sets, wherein each said probe set is capable of hybridizing under stringent or nucleic acid array hybridization conditions to a different respective gene which is differentially expressed in said osteoarthritic cartilage cells relative to said non-osteoarthritic cartilage cells.

4. The nucleic acid array according to claim 3, wherein each said probe set comprises at least 12 polynucleotide probes.

5. The nucleic acid array according to claim 3, wherein the average expression level of each said gene in said osteoarthritic cartilage cells is substantially higher than that in said non-osteoarthritic cartilage cells.

6. The nucleic acid array according to claim 5, further comprising at least 100 polynucleotide probe sets, each of which is capable of hybridizing under stringent or nucleic acid array hybridization conditions to a different respective gene whose average expression level in said non-osteoarthritic cartilage cells is substantially higher than that in said osteoarthritic cartilage cells.

7. The nucleic acid array according to claim 6, wherein said osteoarthritic and non-osteoarthritic cartilage cells are Canis familiars cartilage cells.

8. The nucleic acid array according to claim 1, comprising at least one polynucleotide probe which is capable of hybridizing under stringent or nucleic acid array hybridization conditions to a tiling sequence selected from Table C, or the complement thereof, wherein said tiling sequence has a pattern value of 010, 011, 100 or 101, as shown in Table D.

9. The nucleic acid array according to claim 1, comprising at least 10 polynucleotide probe sets, wherein each said probe set is capable of hybridizing under stringent or nucleic acid array hybridization conditions to a different respective tiling sequence selected from Table C, or the complement thereof, and wherein each said tiling sequence has a pattern value of 010, 011, 100 or 101, as shown in Table D.

10. The nucleic acid array according to claim 1, comprising at least 100 polynucleotide probe sets, wherein each said probe set is capable of hybridizing under stringent or nucleic acid array hybridization conditions to a different respective tiling sequence selected from Table C, or the complement thereof, and wherein each said tiling sequence has a pattern value of 010, 011, 100 or 101, as shown in Table D.

11. A method of screening for drug candidates capable of modulating expression of genes that are differentially expressed in osteoarthritic cartilage cells relative to non-osteoarthritic cartilage cells, comprising the steps of:
(a) preparing a first nucleic acid sample from a vertebrate affected by osteoarthritis;
(b) hybridizing the first nucleic acid sample to a first nucleic acid array as in any one of claims 1-4;
(c) detecting a first set of hybridization signals;
(d) treating the vertebrate with a candidate drug;
(e) repeating steps (a)-(c) with a second nucleic acid sample from the treated vertebrate and a second nucleic acid array identical to the first array to obtain a second set of hybridization signals; and
(f) comparing the first and second sets of hybridization signals, wherein any change in expression level of at least one gene differentially expressed in osteoarthritic cartilage cells relative to non-osteoarthritic cartilage cells identifies the candidate drug as one that modulates expression of said gene.

12. The method according to claim 11, wherein the vertebrate is a canine animal, and the first and second nucleic acid samples are prepared from cartilage tissues of said canine animal.

13. A method of screening for drug candidates capable of modulating expression of genes that are differentially expressed in osteoarthritic cartilage cells relative to non-osteoarthritic cartilage cells, comprising the steps of:
(a) preparing a first nucleic acid sample from a cartilage cell or tissue affected by osteoarthritis;
(b) hybridizing the first nucleic acid sample to a first nucleic acid array as in any one of claims 1-4;
(c) detecting a first set of hybridization signals;
(d) treating the cell or tissue with a candidate drug;
(e) repeating steps (a)-(c) with a second nucleic acid sample from the treated cell or tissue and a second nucleic acid array identical to the first array to obtain a second set of hybridization signals; and
(f) comparing the first and second sets of hybridization signals, wherein any change in expression level of at least one gene differentially expressed in osteoarthritic cartilage cells relative to non-osteoarthritic cartilage cells identifies the candidate drug as one that modulates expression of said gene.

14. A method for detecting gene expression in a sample of interest, comprising:
hybridizing nucleic acid molecules prepared from said sample to a nucleic acid array as in any one of claims 1-4; and
detecting hybridization signals on the nucleic acid array.

15. A nucleic acid array comprising a plurality of polynucleotide probes, wherein each said probe is capable of hybridizing under stringent or nucleic acid array hybridization conditions to a different respective tiling sequence selected from Table C, or the complement thereof.

16. The nucleic acid array according to claim 15, wherein said plurality of polynucleotide probes includes at least 10 probe sets, and each said probe set is capable of hybridizing under stringent or nucleic acid array hybridization conditions to a different respective tiling sequence selected from Table C, or the complement thereof.

17. The nucleic acid array according to claim 15, wherein said plurality of polynucleotide probes includes at least 100 probe sets, and each said probe set is capable of hybridizing under stringent or nucleic acid array hybridization conditions to a different respective tiling sequence selected from Table C, or the complement thereof.

18. A method of making a nucleic acid array, comprising the steps of:
selecting a plurality of polynucleotide probes, each of which is capable of hybridizing under stringent or nucleic acid array hybridization conditions to a different respective gene which is differentially expressed in osteoarthritic cartilage cells relative to non-osteoarthritic cartilage cells; and
attaching the plurality of polynucleotide probes to one or more substrate supports, wherein said osteoarthritic and non-osteoarthritic cartilage cells are derived from the same species.

19. A polynucleotide collection comprising at least one polynucleotide capable of hybridizing under stringent or nucleic acid array hybridization conditions to a parent sequence selected from SEQ ID NOs: 1-12,167, or the complement thereof.

20. A probe array comprising a plurality of probes capable of binding to expression products of genes that are differentially expressed in osteoarthritic cartilage cells relative to non-osteoarthritic cartilage cells, wherein said osteoarthritic cells and said non-osteoarthritic cartilage cells are derived from the same species.

21. The probe array according to claim 20, wherein each said gene encodes a tiling sequence selected from Table C and having a pattern value of 010, 011, 100 or 101.
